(19) United States
(12) Patent Application Publication
(10) Pub. No.: US 2020/0215112 A1
(43) Pub. Date: Jul. 9, 2020

(54) CHIMERIC ANTIGEN RECEPTOR FOR HER2/NEU AND T-CELLS EXPRESSING SAME

(71) Applicant: CTG PHARMA LTD., Rehovot (IL)

(72) Inventors: Debora Rosa BUBLIK, Rehovot (IL); Zelig ESHHAR, Rehovot (IL); Salomon STEMMER, Givat Shmuel (IL); Izhak HAVIV, Rosh Pina (IL); Guy ALLON, Ganei Yohanan (IL); Ido BACHELET, Tel Aviv (IL); Yoav BEN YAACOV, Even Yehuda (IL); Nadav COHEN, Kfar Saba (IL)

(21) Appl. No.: 16/637,503

(22) PCT Filed: Aug. 8, 2018
(86) PCT No.: PCT/IL2018/050877

§ 371 Date: Feb. 7, 2020

Related U.S. Application Data
(60) Provisional application No. 62/542,831, filed on Aug. 9, 2017.

(51) Int. Cl. A61K 35/17; C07K 14/725; C07K 14/705; C07K 14/82; C12N 15/62; A61P 35/00

(52) U.S. Cl. CPC A61K 35/17; C07K 14/7051; A61P 35/00; C07K 14/82; C12N 15/62; C07K 14/70521

(57) ABSTRACT

The present invention provides compositions and methods for improved T-cell therapy of cancer characterized by overexpression of Her2/neu oncogene. Provided are T cells capable of expressing or expressing chimeric antigen receptor (CAR) that specifically recognizes Her2/neu. These T cells have prolonged half-life in the blood circulation and enhanced cytotoxicity, and are used in treatment of a cancer characterized by overexpression of Her2/neu oncogene.

### FIELD OF THE INVENTION

The present invention relates to T cells expressing a chimeric antigen receptor that specifically recognizes Her2 protein and having prolonged half-life in the blood circulation and enhanced cytotoxicity, to use of these CAR T-cells in treatment of a cancer characterized by overexpression of Her2/neu oncogene.

### BACKGROUND OF THE INVENTION

Chimeric antigen receptor T (CAR-T) cell therapy is a newly developed adoptive antitumor treatment. Genetically modified T cells express chimeric antigen receptors, which generally consist of a signaling domain, a transmembrane domain and an extracellular antigen binding domain, typically a single-chain variable fragment (scFv) derived from a monoclonal antibody which gives the receptor specificity for a tumor-associated antigen on a target malignant cell. Upon binding the tumor-associated antigen via the chimeric antigen receptor, the CAR-T cell mounts an immune response that is cytotoxic to the malignant cell. Theoretically, CAR-T cells can specifically localize and eliminate tumor cells by interacting with the tumor-associated antigens (TAAs) expressed on tumor cell surface.

Human Epidermal Growth Factor Receptor 2 (also referred to as Her2/neu or ErbB-2) is a 185 kDa cytoplasmic transmembrane tyrosine kinase receptor. It is encoded by the c-erbB-2 gene and is a member of the HER family (Ross et al., 2003). The HER family normally regulates cell growth and survival, as well as adhesion, migration, differentiation and other cellular responses. Overexpression and/or amplification of Her2/neu is observed in the development of a variety of solid cancers including breast, gastric, stomach, colorectal, ovarian, pancreatic, endometrial and non-small cell lung cancers.

A number of Her2/neu targeting therapies have been approved for treatment of Her2/neu positive tumors. HERCEPTIN° (trastuzumab) is approved for the treatment of metastatic breast cancer in combination with TAXOL® (paclitaxel) and alone for the treatment of Her2/neu positive breast cancer in patients who have received one or more chemotherapy courses for metastatic disease.

US 2016/060724 describes chimeric transmembrane immunoreceptors (CAR) which include an extracellular domain targeted to Her2/neu, a transmembrane region, and a costimulatory domain.

Eshhar and co-workers (Globerson-Levin et al., Molecular Therapy, 2014, 22 (5), 1029-1038) studied the ability of T cells expressing a chimeric antibody-based receptor (CAR) to offer therapeutic benefit for breast cancer induced by ErbB-2. Globerson-Levin et al. found that repeated administration of CAR-modified T cells is required to eliminate spontaneously developing mammary cancer.

The constant search for more potent therapies has led to the generation of more complex structures of chimeric antigen receptors including use of multiple intracellular signaling elements (Yu et al. J. Hematology and Oncology, 2017, 10:78; Song et al., Cancer Res., 2011, 71(13): 4617-4627; Sadelain et al., Cancer Discov., 2013, 3(4), 388-398).

WO 2017/079694 discloses Chimeric Antigen Receptors targeting HER-2, wherein the receptors have multiple amino acid modifications.

There is an unmet medical need for improved CAR T-cell systems that overcome the drawbacks that currently prevent utilization of the systems and their development into effective means of in vivo treatment.

### SUMMARY OF THE INVENTION

The present invention provides compositions and methods for improved T cell therapy of cancer using a chimeric antigen receptor that is designed to recognize Her2/neu oncogene and provides a prolonged persistence in the blood and enhanced cytotoxicity of the T cells.

According to one aspect, the present invention provides a nucleic acid encoding a chimeric antigen receptor (CAR), wherein the CAR comprises in order from N-Terminus to C-Terminus:
(i) a leader peptide;
(ii) a Her2 binding domain comprising a light chain variable region (V_{L}) having amino acid sequence SEQ ID NO: 11 and a heavy chain variable region (V_{H}) having amino acid sequence SEQ ID NO: 12, wherein the V_{L} and the V_{H} are linked by a peptide linker;
(iii) a hinge region selected from the hinge region of CD28 and CD8,
(iv) a transmembrane (TM) domain selected from the TM domain of CD28 and ICOS;
(v) a first co-stimulatory (CS) domain selected from the co-stimulatory domain of CD28 and ICOS;
(vi) a second co-stimulatory domain which is the co-stimulatory domain of human 4-1BB receptor having amino acid sequence SEQ ID NO: 9; and
(vii) an activation domain selected from the human activation domain CD3ζ having the amino acid sequence SEQ ID NO: 21 and the activation domain of human Fcγ receptor (FcγR) having amino acid sequence SEQ ID NO: 10.

According to one embodiment, the nucleic acid is an isolated nucleic acid.

According to some embodiments, the leader peptide has an amino acid sequence selected from SEQ ID NO: 13 and SEQ ID NO: 26. According to one embodiment, the leader peptide consists of an amino acid sequence selected from SEQ ID NO: 13 and SEQ ID NO: 26.

According to some embodiments, the peptide linker consists of 10-30 amino acids. According to one embodiment, the peptide linker has amino acid sequence SEQ ID NO: 14. According to another embodiment, the peptide linker consists of amino acid sequence SEQ ID NO: 14.

According to some embodiments, the TM domain and the first SC domain together have amino acid sequence SEQ ID NO: 24. According to one embodiment, the TM domain and the first SC domain together consist of amino acid sequence SEQ ID NO: 24.

According to some embodiments, the TM and the SC domains together have amino acid sequence SEQ ID NO: 20. According to one embodiment, the TM domain and the first SC domain together consist of amino acid sequence SEQ ID NO: 20.

According to some embodiments, the activation domain has an amino acid sequence SEQ ID NO: 21. According to one embodiments, the activation domain consists of amino acid sequence SEQ ID NO: 21.

According to one embodiment, the hinge region has an amino acid sequence selected from SEQ ID NO: 22 and SEQ ID NO: 19. According to one embodiment, the hinge region consists of amino acid sequence selected from SEQ ID NO: 22 and SEQ ID NO: 19.

According to any one of the above embodiments, the Her2 binding domain is a single-chain variable fragment (ScFv). According to one embodiment, the Her2 binding domain has amino acid sequence SEQ ID NO: 23. According to another embodiment, the Her2 binding domain consists of amino acid sequence SEQ ID NO: 23.

According to some embodiments, the nucleic acid of the present invention further comprises a co-stimulatory domain selected from a CS domain of CD80, OX40 CD154, CD27, and CD244.

According to some embodiments, the present invention provides an isolated nucleic acid encoding the CAR of the present invention, wherein the Her2 binding domain has amino acid sequence SEQ ID NO: 23 and the activation domain has amino acid sequence SEQ ID NO: 21. According to one embodiment, the hinge region has amino acid sequence SEQ ID NO: 19. According to another embodiment, the leader peptide has amino acid sequence SEQ ID NO: 26. According to certain embodiments, the TM and first SC domain have together an amino acid sequence selected from SEQ ID NO: 24 and SEQ ID NO: 20. According to one embodiment, the CAR comprises amino acid sequence SEQ ID NO: 28. According alternative embodiment, the CAR comprises amino acid sequence SEQ ID NO: 29.

According to some embodiments, the present invention provides an isolated nucleic acid encoding the CAR of the present invention, wherein the Her2 binding domain has amino acid sequence SEQ ID NO: 23, the hinge region has the amino acid sequence SEQ ID NO: 22 and the TM and the first SC domain have together amino acid sequence SEQ ID NO: 24. According to some embodiments, the leader peptide has the amino acid sequence SEQ ID NO: 13. According to one embodiment, the CAR comprises an amino acid sequence SEQ ID NO: 15. According to another embodiment, the CAR consists of amino acid sequence SEQ ID NO: 15. According to an alternative embodiment, the CAR comprises an amino acid sequence SEQ ID NO: 27. According to a further embodiment, the CAR consists of amino acid sequence SEQ ID NO: 27.

According to some embodiments, the present invention provides an isolated nucleic acid comprising a nucleic acid sequence selected from SEQ ID NO: 27, 28, 29 and 40. According to other embodiments, the present invention provides an isolated nucleic acid consisting of a nucleic acid sequence selected from SEQ ID NO: 27, 28, 29 and 40.

According to another aspect, the present invention provides a nucleic acid construct comprising the nucleic acid of the present invention, operably linked to a promoter.

According to a further aspect, the present invention provides a vector comprising the nucleic acid of the present invention. According to one embodiment, the nucleic acid is an isolated nucleic acid. According to some embodiments, the present invention provides a vector comprising the nucleic acid construct of the present invention.

According to one aspect, the present invention provides a genetically modified T cell (CAR-T cell) comprising the isolated nucleic acid of the present invention. According to another embodiment, the present invention provides a genetically modified T cell comprising the vector of the present invention. According to one embodiment, the CAR-T of the present invention is capable of expressing the CAR of the present invention. According to another embodiment, the CAR-T of the present invention expresses the CAR of the present invention.

According to some aspects, the present invention provides a population of T cells comprising a plurality of the genetically modified CAR T cells of the present invention. According to some embodiments, the population is an enriched population comprising at least 30% genetically modified T cells. According to one embodiment, the CAR-T cells of the present invention exhibit at least one of higher cell surface expression, increased cytotoxicity, higher CAR-T cell proliferation upon activation, higher expansion, higher persistence, and lower exhaustion rate in comparison to known N29 CAR T-cells. According to one embodiment, at least 20% of the administered said T cells upon intravenous administration persist in the serum at least 4 weeks after administration.

According to another aspect, the present invention provides a pharmaceutical composition comprising the genetically modified T cells of the present invention, and a pharmaceutically acceptable excipient. According to one embodiment, the pharmaceutical composition is for use in treating cancer. According to one embodiment, the cancer is selected from a breast cancer, ovary cancer, gastric cancer, glioblastoma, osteosarcoma, and medulloblastoma. According to another embodiment, the cancer is a breast cancer. According to certain embodiments, the breast cancer is a Her2 positive cancer. In some embodiments, the breast cancer has a score of 2+ or 3+ in a Her2 immunohistochemistry test. According to other embodiments, wherein the breast cancer has a score of 1+ in Her2 immunohistochemistry test.

According to a further aspect, the present invention provides a method of treating cancer in a subject in need thereof, comprising administering a therapeutically effective amount of the genetically modified T cells of the present invention.

These and further aspects and embodiments of the invention will become apparent in conjunction with the figures the detailed description and the examples that follow. It is to be expressly understood, however, that any description, figure, example, etc. is provided for the purpose of illustration and description only and is by no means intended to define the limits the invention.

### DETAILED DESCRIPTION OF THE INVENTION

According to one aspect the present invention provides a nucleic acid encoding a chimeric antigen receptor (CAR), wherein the CAR comprises in order from N-Terminus to C-Terminus (i) a leader peptide, (ii) a Her2 binding domain comprising a light chain variable region (V_{L}) and a heavy chain variable region (V_{H}), (iii) a hinge region selected from the hinge region of CD28 and CD8, (iv) a transmembrane (TM) domain selected from the TM domain of CD28 and ICOS; (v) a first co-stimulatory (CS) domain selected from the CS domain of CD28 and ICOS; (vi) a second co-stimulatory domain being the CS domain of human 4-1BB receptor having the amino acid sequence SEQ ID NO: 9; and (vii) an activation domain selected from the activation domain CD3ζ having the amino acid sequence SEQ ID NO: 12 and the activation domain of human Fcγ receptor (FcγR) having the amino acid sequence SEQ ID NO: 10, wherein the V_{L} comprises three complementarity determining regions (CDRs) within the amino acid sequence SEQ ID NO: 11 and the V_{H} comprises three CDRs within the amino acid sequence SEQ ID NO: 12. As it is well known in the art the CDRs may defined according to different methods such as Kabat and IMGT methods. According to one embodiment, the CDRs of the V_{L} have amino acid sequences SEQ ID NOs: 2, 3, and 4, and the CDRs of the V_{H} have amino acid sequences SEQ ID NO: 5, 6 and 7. According to one embodiment, the V_{L} has amino acid sequence SEQ ID NO: 11. According to another embodiment, the V_{H} has amino acid sequence SEQ ID NO: 12. According to a further embodiment, the V_{L} has amino acid sequence SEQ ID NO: 11 and the V_{H} has amino acid sequence SEQ ID NO: 12. Thus, in one aspect the present invention provides a nucleic acid encoding a chimeric antigen receptor (CAR), wherein the CAR comprises in order from N-Terminus to C-Terminus:
(i) a leader peptide;
(ii) a Her2 binding domain comprising a light chain variable region (V_{L}) having amino acid sequence SEQ ID NO: 11 and a heavy chain variable region (V_{H}) having amino acid sequence SEQ ID NO: 12, wherein the V_{L} and the V_{H} are linked by a peptide linker;
(iii) a hinge region selected from the hinge region of CD28 and CD8,
(iv) a transmembrane (TM) domain selected from the TM domain of CD28 and TM domain of ICOS;
(v) a first co-stimulatory (CS) domain selected from the co-stimulatory domain of CD28 and CS domain of ICOS;
(vi) a second co-stimulatory domain which is the co-stimulatory domain of human 4-1BB receptor having amino acid sequence SEQ ID NO: 9; and
(vii) an activation domain selected from the human activation domain CD3ζ having the amino acid sequence SEQ ID NO: 21 and the activation domain of human Fcγ receptor (FcγR) having amino acid sequence SEQ ID NO: 10.
   According to some embodiments, the nucleic acid is an isolated nucleic acid. According to one embodiment, the Her2 binding domain binds specifically to Her2 protein.

The term “nucleic acid” encompasses DNA, RNA, single stranded or double stranded and chemical modifications thereof. The terms “nucleic acid” and “polynucleotide” are used herein interchangeably herein. In one embodiment, the nucleic acid is a DNA polynucleotide.

The term “sequence” as used herein refers to an amino acid sequence (for peptides and proteins) and to nucleic acid sequence (for DNA and RNA). This term also contemplates the analogs of a peptide or protein sequences and homologs of a nucleic acid sequence having at least 90% identity to the original (parent) sequence. According to some embodiments, the homolog or analog has 90% to 99%, 91% to 98%, 92% to 97%, 93% to 96% or 94% to 95% identity to the original sequence. As such the sequences SEQ ID NOs: 9-46 encompass also analogs and homologs thereof having at least 90%, at least 92% at least 95%, at least 98% or at least 99% identity to the original sequence.

According to any one of the aspects and embodiments of the present invention, the terms “peptide comprising the amino acid sequence set forth in SEQ ID NO: X”, “peptide comprising SEQ ID NO: X” and “peptide having SEQ ID NO: X” are used herein interchangeably. The terms “peptide consisting of the amino acid sequence set forth in SEQ ID NO: X”, “peptide consisting of SEQ ID NO: X” and “peptide of SEQ ID NO: X” are used herein interchangeably. These statements hold also for proteins sequences.

The same rule holds for nucleic acid sequence. Thus the terms “nucleic acid comprising the nucleic acid sequence set forth in SEQ ID NO: X”, “nucleic acid comprising SEQ ID NO: X” and “nucleic acid having SEQ ID NO: X” are used herein interchangeably. The terms “nucleic acid consisting of the nucleic acid sequence set forth in SEQ ID NO: X”, “nucleic acid consisting of SEQ ID NO: X” and “nucleic acid of SEQ ID NO: X” are used herein interchangeably.

The term “peptide” encompasses also the term “peptide analog”. The term “peptide analog”, “analog” and “sequence analog” are used herein interchangeably and refer to an analog of a peptide having at least 90% sequence identity with the original peptide, wherein the analog retains the activity of the original peptide. Thus, the terms “analog” and “active analog” may be used interchangeably. The term “analog” refer to a peptide which contains substitutions, rearrangements, deletions, additions and/or chemical modifications in the amino acid sequence of the parent peptide. According to some embodiments, the peptide analog has at least 95% or at least 98% sequence identity to the original peptide. According to one embodiment, the analog has 90% to 99%, 91% to 98%, 92% to 97%, 93% to 96% or 94% to 95% identity to the original sequence to the original peptide. According to some embodiments, the analog of the present invention comprises the sequence of the original peptide or protein in which 1, 2, 3, 4, or 5 substitutions were made.

The substitutions of the amino acids may be conservative or non-conservative substitution. The non-conservative substitution encompasses substitution of one amino acid by any other amino acid.

The term “conservative substitution” as used herein denotes the replacement of an amino acid residue by another, without altering the overall conformation and biological activity of the peptide, including, but not limited to, replacement of an amino acid with one having similar properties (such as, for example, polarity, hydrogen bonding potential, acidic, basic, shape, hydrophobic, aromatic, and the like). Amino acids with similar properties are well known in the art. For example, according to one table known in the art, the following six groups each contain amino acids that are conservative substitutions for one another: (1) Alanine (A), Serine (S), Threonine (T); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); and (6) Phenylalanine (F), Tyrosine (Y), Tryptophan (W).

The terms “homolog” and “polynucleotide homolog” are used herein interchangeably and refer to a polynucleotide having at least 90% sequence identity to the original (parent) polynucleotide. The homolog may include mutations such as deletion, addition or substitution such that the mutations do not change the open reading frame and the polynucleotide encodes a peptide or a protein having substantially similar structure and function as a peptide or a protein encoded by the parent polynucleotide. According to some embodiments, the homologs are conservative homologs. The term “conservative homologs” as used herein refers to homologs in which a change of one or more nucleotides in a given codon position results in no alteration in the amino acid encoded at that position. Thus, the peptide or the protein encoded by the conservative homologs has 100% sequence identity to the peptide or the protein encoded by the parent polynucleotide. According to some embodiments, the homolog is a non-conservative homolog encoding to a peptide or a protein being a conservative analog of the peptide of the protein encoded by the parent polynucleotide. According to some embodiments, the homolog has at least 95%, at least 98% or at least 99% sequence identity to the original polynucleotide.

The term “isolated nucleic acid” as used herein denotes that the nucleic acid is essentially free of other cellular components with which it is associated in the cell. It can be, for example, a homogeneous state and may be dry or in the state of a solution, such as aqueous solution. Purity and homogeneity are typically determined using analytical chemistry techniques such as polyacrylamide gel electrophoresis or high performance liquid chromatography.

The term “encoding” refers to the ability of a nucleotide sequence to code for one or more amino acids. The term does not require a start or stop codon. An amino acid sequence can be encoded in any one of six different reading frames provided by a polynucleotide sequence and its complement.

The terms “chimeric antigen receptor” or “CAR” are used herein interchangeably and refer to engineered receptors, which are grafted onto cells. In general, a CAR comprises an extracellular domain (extracellular part) comprising the antigen binding domain, a transmembrane domain and an intracellular domain.

The extracellular domain comprises an antigen binding domain and optionally a spacer or hinge region.

The antigen binding domain of the CAR targets a specific antigen. The targeting regions may comprise full length heavy chain, Fab fragments, or single chain variable fragment (scFv). The antigen binding domain can be derived from the same species or a different species for or in which the CAR is used. In one embodiment, the antigen binding domain is scFv.

The extracellular spacer or hinge region of a CAR is located between the antigen binding domain and a transmembrane domain. Extracellular spacer domains may include, but are not limited to, Fc fragments of antibodies or fragments or derivatives thereof, hinge regions of antibodies or fragments or derivatives thereof, CH2 regions of antibodies, CH3 regions of antibodies, accessory proteins, artificial spacer sequences or combinations thereof.

The term “transmembrane domain” refers to the region of the CAR, which crosses or bridges the plasma membrane. The transmembrane domain of the CAR of the present invention is the transmembrane region of a transmembrane protein, an artificial hydrophobic sequence or a combination thereof.

The terms “specifically binds” or “specific for” with respect to an antigen-binding domain of an antibody, of a fragment thereof or of a CAR refers to an antigen-binding domain, which recognizes and binds to a specific antigen, but does not substantially recognize or bind other molecules in a sample. The term encompasses that the antigen-binding domain binds to its antigen with high affinity and binds to other antigens with low affinity. An antigen-binding domain that binds specifically to an antigen from one species may bind also to that antigen from another species. This cross-species reactivity is not contrary to the definition of that antigen-binding domain as specific. An antigen-binding domain that specifically binds to an antigen may bind also to different allelic forms of the antigen (allelic variants, splice variants, isoforms etc.). This cross reactivity is not contrary to the definition of that antigen-binding domain as specific.

The term “peptide linker” relates to any peptide capable of connecting two variable domains with its length depending on the kinds of variable domains to be connected.

The term “leader peptide” and “signal peptide” are used herein interchangeably and refer to a peptide that lead the protein towards a membrane and in particular to the cell membrane.

The terms “Her2/neu”, “Her2” and “HER2” are used herein interchangeably and refer to a human oncogene and a member of the human adenocarcinoma-associated growth factor receptor family, which is overexpressed on several human cancers and is known also as erbB-2 and CD340. Thus, according to any one of the embodiments of the present invention, the Her2/neu binding domain is a domain that binds human Her2/neu.

The terms “light chain variable region” and “V_{L}” are used herein interchangeably and refer to a light chain variable region of an antibody capable of binding to Her2 receptor.

The terms “heavy chain variable region” and “V_{H}” are used herein interchangeably and refer to a heavy chain variable region of an antibody capable of binding to Her2 receptor.

According to any one of the embodiments of the present invention, the terms “having the sequence”, “has the sequence”, “comprising the sequence”, and “comprises the sequence” are used herein interchangeably and also have the meaning of “consisting of” the sequence and may be substituted by it.

The term “CD28” refers to cluster of differentiation 28 protein. In some embodiments, the CD28 is a human CD28.

The term “CD8” refers to cluster of differentiation 8 protein being a transmembrane glycoprotein and serving as a co-receptor for the T cell receptor. According to one embodiment, the CD8 is a human CD8.

The terms “ICOS” and “Inducible T-cell COStimulator” refer to CD278 which is a CD28-superfamily costimulatory molecule. According to one embodiment, the ICOS is a human ICOS.

The term “4-1BB” refers to a CD137 protein which is a member of the tumor necrosis factor receptor family and has costimulatory activity for activated T cells. According to one embodiment, 4-1BB is a human 4-1BB.

The term “CD3ζ” refers to a ζ chain of CD3 (cluster of differentiation 3) T cell co-receptor participating in activation of both the cytotoxic and helper T cells. According to one embodiment, CD3ζ comprises an immunoreceptor tyrosine-based activation motif (ITAM). According to one embodiment, the CD3ζ is human CD3ζ. CD3ζ is sometimes also referred as CD247.

The term “FcγR” refers to Fc gamma receptors, which generate signals within their cells through ITAM. These are immunoglobulin superfamily receptors that are found on various innate as well as adoptive immune cells, where the extracellular part binds IgGs the activation signal is transduced through two ITAMs located on its cytoplasmic tail.

According some embodiments, the leader peptide is an Ig kappa chain signal peptide. According to some embodiments, the leader peptide has the sequence SEQ ID NO: 13.

According to other embodiments, the leader peptide is the signal peptide of CD8α. According to one embodiment, the leader peptide has amino acid sequence SEQ ID NO: 26.

According to some embodiments, the V_{L} and V_{H} are separated by a peptide linker consisting of 10-30 amino acids. According to another embodiment, the peptide linker consists of 12 to 28, 13 to 25, 14 to 22 of 15 to 20 amino acids. According to another embodiment, the peptide linker consists of 10 to 20, 11 to 19, 12 to 18, 13 to 17, 14 to 16 or to 15 amino acids. According to another embodiment, the linker comprises the amino acid sequence SEQ ID NO: 14. According to a further embodiment, the linker consists of amino acid sequence SEQ ID NO: 14.

According to one embodiment, the TM domain of the CAR of the present invention is the TM of CD28 protein. According to one such embodiment, the TM comprises amino acid sequence SEQ ID NO: 18. According to another embodiment, the TM domain of the CAR of the present invention is the TM of ICOS protein.

According to some embodiments, the first co-stimulatory domain is the CS domain of CD28 protein. According to certain embodiments, the first co-stimulatory domain is the CS domain of ICOS protein.

According to one embodiment, the TM and the first CS domain have together (jointly) amino acid sequence SEQ ID NO: 24.

According to another embodiment, the TM and the first CS domain have together amino acid sequence SEQ ID NO: 20

According to some embodiments, the hinge region is the hinge region of CD28. According to one embodiment, the hinge region has amino acid sequence SEQ ID NO: 22.

According to other embodiments, the hinge region is the hinge region of CD8. According to one embodiment, the hinge region has amino acid sequence SEQ ID NO: 19.

According to one embodiment, the activation domain has amino acid sequence SEQ ID NO: 21

According to another embodiment, the activation domain has amino acid sequence SEQ ID NO: 10.

According to any one of the above embodiments, the Her2 binding domain is a single-chain variable fragment (ScFv), i.e. a structure comprising V_{H} and V_{L} linked as a single chain. According to one embodiment, the V_{H} is located N-terminally to V_{L}. According to another embodiment, the V_{L} is located N-terminally to V_{H}. According to some embodiments, the V_{L} and V_{H} are separated by a peptide linker. According to one embodiment, the peptide linker consists of 10 to 20, 11 to 19, 12 to 18, 13 to 17, 14 to 16 or to 15 amino acids. According to another embodiment, the linker comprises amino acid sequence SEQ ID NO: 14. According to a further embodiment, the linker consists of the consists of amino acid sequence SEQ ID NO: 14. According to one embodiment, the Her2 binding domain comprises the amino acid sequence SEQ ID NO: 23. According to another embodiment, the Her2 binding domain consists of amino acid sequence SEQ ID NO: 23. According to any one of the above embodiments, the Her2 binding domain binds specifically to Her2 protein.

According to some alternative embodiments, the isolated nucleic acid encodes the CAR of the present invention, said CAR comprises a cytoplasmic signaling (co-stimulatory) domain of a protein selected from CD134 (TNFRSF4, OX40), CD154 (CD40L), ICOS (CD278), CD27, DAP10 and CD244 (2B4) instead of the cytoplasmic signaling domain of human 4-1BB receptor. In other alternative embodiments, the isolated nucleic acid encodes a CAR comprising at least one signaling domain of a protein selected from CD80, DAP10, CD134 (TNFRSF4, OX40), CD154 (CD40L), (CD278), CD27, and CD244 (2B4) in addition to the co-stimulatory signaling domain of human 4-1BB. In such embodiments, the additional signaling domain is located C-terminally or N-terminally to the signaling domain of human 4-1BB. According to one embodiment, the CAR of the present invention comprises 4-1BB CS domain and further comprises an additional CS domain of a protein selected from CD80, OX40, CD154, CD27, and CD244. According to one embodiment, the CAR of the present invention further comprises a CS domain of CD80. According to one embodiment, the CAR of the present invention comprises a CS domain of CD80 located C-terminally to 4-1BB CS domain. According to another embodiment, the CS domain of CD80 is located N-terminally to 4-1BB.

According to one embodiment, the present invention provides an isolated nucleic acid encoding the CAR of the present invention in which the Her2 binding domain has amino acid sequence SEQ ID NO: 23 and the activation domain has amino acid sequence SEQ ID NO: 21. According to one embodiment, the hinge region has amino acid sequence SEQ ID NO: 19. According to another embodiment, the leader peptide has amino acid sequence SEQ ID NO: 26. According to yet another embodiment, the hinge region has amino acid sequence SEQ ID NO: 19 and the leader peptide has amino acid sequence SEQ ID NO: 26. According to one such embodiment, the TM and first SC domain have together amino acid sequence SEQ ID NO: 24. According to another such embodiment, the TM and first SC domain have together amino acid sequence SEQ ID NO: 20. According to one embodiment, the present invention provides an isolated nucleic acid encoding a chimeric antigen receptor having the amino acid sequence SEQ ID NO: 28. According to another embodiment, the CAR consists of the amino acid sequence SEQ ID NO: 28. According to a further embodiment, the present invention provides the isolated nucleic acid encoding a chimeric antigen receptor having amino acid sequence SEQ ID NO: 29. According to another embodiment, the CAR consists of the amino acid sequence SEQ ID NO: 29.

According to alternative embodiments, the present invention provides an isolated nucleic acid encoding the CAR of the present invention in which the Her2 binding domain has amino acid sequence SEQ ID NO: 23, the hinge region has amino acid sequence SEQ ID NO: 19 and the activation domain has amino acid sequence SEQ ID NO: 21. According to some embodiments, the leader peptide has amino acid sequence SEQ ID NO: 13.

According to one embodiment, the present invention provides an isolated nucleic acid encoding the CAR of the present invention in which Her2 binding domain has the amino acid sequence SEQ ID NO: 23, the hinge region has the amino acid sequence SEQ ID NO: 22 and the TM and the first SC domain have together amino acid sequence SEQ ID NO: 24. According to some such embodiments, the leader peptide has the amino acid sequence SEQ ID NO: 13. According to one embodiment, the activation domain has amino acid sequence SEQ ID NO: 21. According to an alternative embodiment, the activation domain has amino acid sequence SEQ ID NO: 10. According to one embodiment, the present invention provides the isolated nucleic acid encoding a CAR having amino acid sequence SEQ ID NO: 15. According to another embodiment, the CAR consists of the amino acid sequence SEQ ID NO: 15. According to a further embodiment, the present invention provides the isolated nucleic acid encoding a CAR having the amino acid sequence SEQ ID NO: 27. According to yet another embodiment, the CAR consists of the amino acid sequence SEQ ID NO: 27.

According to any one of the above embodiments, the CAR of the present invention further comprises at least one copy of at least one marker located N-terminally to the hinge region.

The terms “marker”, “tag” and “label” are used herein interchangeably and refer to an amino acid sequence which is detectable by any known method and being used to facilitate purification/isolation/detection of the protein or entity to which the tag is bound or into which it is integrated. Non-limiting examples for the tag are strep-tag II, Flag tag, HIS-tag, biotin, myc, Vesicular Stomatitis Viral Glycoprotein (VSV-G) tag,

According to some embodiments, the Her2/neu binding domain, the leader peptide such as Ig kappa chain signal peptide, the marker and the hinge of the hinge and transmembrane domain (the first 18 N-terminal amino acids) form an extracellular domain of the CAR.

According to one embodiment, the marker is a strep-tag II marker having the sequence SEQ ID NO: 16. According to another embodiment, the marker is a Flag marker having the sequence SEQ ID NO: 17.

According to some embodiments, the CAR comprises one copy of the marker, such as strep-tag II. According to another embodiment, the CAR comprises a plurality of copies of such markers. According to one embodiment, CAR comprises 2 to 10, 3 to 9, 4 to 8, 5 to 7 or to 6 markers, e.g. strep-tag II markers.

According to another embodiment, the CAR comprises two or more different markers, e.g. strep-tag II and Flag tag. According to another embodiment, the CAR comprises one or more copies for each one of said different markers.

According to some embodiments, when a plurality of markers is present, either multiple copies of one marker, one copy for each one of two or more different markers or multiple copies for each one of two or more different markers, the markers may be organized in any possible arrangement.

In one embodiment, all markers are located in one locus or array. In some embodiments, the markers are linked with or without a peptide spacer.

According to another embodiment, the one or more markers is placed on the C-terminus of the leader peptide such as Ig kappa chain signal peptide, and/or on the N-terminus or the C-terminus of V_{L}, and/or on the N-terminus or the C-terminus of V_{H}, or between V_{L} and V_{H} as a peptide linker. According to one embodiment, the marker, such as strep-tag II or Flag marker, is placed C-terminally to V_{H} domain, with or without a peptide spacer.

According to some embodiments, the CAR comprises one or more strep-tag II markers. According to one embodiment, the CAR comprises 2 to 10, 3 to 9, 4 to 8, 5 to 7 or to 6 strep-tag II markers. According to some embodiments, CAR comprises 2 or more strep-tag II markers located in one array or in different location of the extracellular domain of the CAR.

According to some embodiments, the nucleic acid encoding the V_{L} having amino acid sequence SEQ ID NO: 11 comprises nucleic acid sequence SEQ ID NO: 30. According to another embodiment, the nucleic acid encoding the V_{H} having amino acid sequence SEQ ID NO: 12 comprises nucleic acid sequence SEQ ID NO: 31. According to one embodiment, the nucleic acid encoding the peptide linker having amino acid sequence SEQ ID NO: 14 comprises nucleic acid sequence SEQ ID NO: 32. According another embodiment, the nucleic acid encoding the Her2 binding domain comprises or consists of nucleic acid sequence SEQ ID NO: 33. According to some embodiments, the nucleic acid encoding 4-1BB co-stimulatory domain comprises nucleic acid sequence SEQ ID NO: 34. According to yet another embodiment, the nucleic acid encoding the TM and the SC domain of CD28 comprises nucleic acid sequence SEQ ID NO: 35. According to a further embodiment, the nucleic acid encoding the TM and the SC domain of ICOS comprises nucleic acid sequence SEQ ID NO: 42. According to one embodiment, the nucleic acid encoding the activation domain CD3ζ comprises nucleic acid sequence SEQ ID NO: 36. According to another embodiment, the nucleic acid encoding the activation domain of FcγR comprises nucleic acid sequence SEQ ID NO: 37. According to one embodiment, the nucleic acid encoding the leader peptide having amino acid sequence SEQ ID NO: 13 comprises nucleic acid sequence SEQ ID NO: 38. According to certain embodiments, the nucleic acid encoding the leader peptide having amino acid sequence SEQ ID NO: 26 comprises nucleic acid sequence SEQ ID NO: 39. According to yet another embodiment, the nucleic acid encoding the hinge region having amino acid sequence SEQ ID NO: 22 comprises nucleic acid sequence SEQ ID NO: 40. According to one embodiment, the nucleic acid encoding the hinge region having amino acid sequence SEQ ID NO: 19 comprises nucleic acid sequence SEQ ID NO: 41. According to some embodiments, the isolated nucleic acid of the present invention comprises combination of the above nucleic acids and said combination encodes the CAR of the present invention. According to some embodiments, the nucleic acids of the present invention are codon optimized nucleic acids. According to some embodiments, codon optimized nucleic acids refer to nucleic acids optimized for humans and/or for improved expression, e.g. in terms of codon usage (considering rare tRNAs for example).

According to one embodiment, the isolated nucleic acid of the present invention comprises the nucleic acid sequence SEQ ID NO: 43. According to another embodiment, the isolated nucleic acid of the present invention comprises the nucleic acid sequence SEQ ID NO: 44. According to a further embodiment, the isolated nucleic acid of the present invention comprises the nucleic acid sequence SEQ ID NO: 45.

According to yet another embodiment, the isolated nucleic acid of the present invention comprises the nucleic acid sequence SEQ ID NO: 46. As defined herein above, the term comprises encompasses also the term consisting. Thus, according to one embodiment, the nucleic acid consists of nucleic acid sequence selected from SEQ ID NO: 43, 44, 45, and 46.

As defined herein above, the term sequence encompasses also the homologs of that sequence having at least 90% identity to the original sequence. Thus, the nucleic acid of the present invention comprises or consists of a homolog of a nucleic acid sequence selected from SEQ ID NO: 43, 44, 45, and 46. According to one embodiment, the homolog has at least 92%, at least 95% or at least 98% identity to the original sequence.

According to a further aspect, the present invention provides a nucleic acid construct comprising the nucleic acid of the present invention operable linked to a promoter.

The term “construct” as used herein refers to an artificially constructed segment of a nucleic acid. It can be isolated or integrated into another nucleic acid molecule. According to one embodiment, the construct is a DNA construct. Accordingly, a “recombinant DNA construct” is produced by laboratory methods.

The terms “operably linked”, “operatively linked”, “operably encodes”, and “operably associated” are used herein interchangeably and refer to the functional linkage between a promoter and nucleic acid sequence, wherein the promoter initiates transcription of RNA corresponding to the DNA sequence. A heterologous DNA sequence is “operatively associated” with the promoter in a cell when RNA polymerase which binds the promoter sequence transcribes the coding sequence into mRNA which then in turn is translated into the protein encoded by the coding sequence.

The term “promoter” as used herein refers to a regulatory sequence that initiates transcription of a downstream nucleic acid. The term “promoter” refers to a DNA sequence within a larger DNA sequence defining a site to which RNA polymerase may bind and initiate transcription. A promoter may include optional distal enhancer or repressor elements. The promoter may be either homologous, i.e., occurring naturally to direct the expression of the desired nucleic acid, or heterologous, i.e., occurring naturally to direct the expression of a nucleic acid derived from a gene other than the desired nucleic acid. A promoter may be constitutive or inducible. A constitutive promoter is a promoter that is active under most environmental and developmental conditions. An inducible promoter is a promoter that is active under environmental or developmental regulation. Promoters may be derived in their entirety from a native gene, may comprise a segment or fragment of a native gene, or may be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. It is further understood that the same promoter may be differentially expressed in different tissues and/or differentially expressed under different conditions.

According to one embodiment, the nucleic acid construct comprises a nucleic acid operably linked to a promoter wherein said nucleic acid encodes a chimeric antigen receptor (CAR) of the present invention, wherein the CAR comprises in order from N-Terminus to C-Terminus:
(i) a leader peptide;
(ii) a Her2 binding domain comprising a light chain variable region (V_{L}) having amino acid sequence SEQ ID NO: 11 and a heavy chain variable region (V_{H}) having amino acid sequence SEQ ID NO: 12, wherein the V_{L} and the V_{H} are linked by a peptide linker;
(iii) a hinge region selected from the hinge region of CD28 and CD8,
(iv) a transmembrane (TM) domain selected from the TM domain of CD28 and ICOS;
(v) a first co-stimulatory (CS) domain selected from the co-stimulatory domain of CD28 and ICOS;
(vi) a second co-stimulatory domain which is the co-stimulatory domain of human 4-1BB receptor having amino acid sequence SEQ ID NO: 9; and
(vii) an activation domain selected from the human activation domain CD3ζ having the amino acid sequence SEQ ID NO: 21 and the activation domain of human Fcγ receptor (FcγR) having amino acid sequence SEQ ID NO: 10.

According to one embodiment, the nucleic acid construct comprises a nucleic acid encoding a CAR of the present invention in which the Her2 binding domain has amino acid sequence SEQ ID NO: 23 and the activation domain has amino acid sequence SEQ ID NO: 21. According to one embodiment, the hinge region has amino acid sequence SEQ ID NO: 19. According to another embodiment, the leader peptide has amino acid sequence SEQ ID NO: 26. According to yet another embodiment, the hinge region has amino acid sequence SEQ ID NO: 19 and the leader peptide has amino acid sequence SEQ ID NO: 26. According to one such embodiment, the TM and first SC domain have together amino acid sequence SEQ ID NO: 24. According to another such embodiment, the TM and first SC domain have together amino acid sequence SEQ ID NO: 20. According to one embodiment, the nucleic acid construct comprises a nucleic acid encoding a chimeric antigen receptor having the amino acid sequence SEQ ID NO: 28. According to another embodiment, the CAR consists of the amino acid sequence SEQ ID NO: 28. According to a further embodiment, the nucleic acid construct comprises a nucleic acid encoding a chimeric antigen receptor having the amino acid sequence SEQ ID NO: 29. According to another embodiment, the CAR consists of the amino acid sequence SEQ ID NO: 29.

According to alternative embodiments, the nucleic acid construct comprises a nucleic acid encoding a CAR of the present invention in which the Her2 binding domain has amino acid sequence SEQ ID NO: 23, the hinge region has amino acid sequence

SEQ ID NO: 19 and the activation domain has amino acid sequence SEQ ID NO: 21. According to some embodiments, the leader peptide has amino acid sequence SEQ ID NO: 13.

According to one embodiment, the nucleic acid construct comprises a nucleic acid encoding a CAR in which wherein Her2 binding domain has the amino acid sequence SEQ ID NO: 23, the hinge region has the amino acid sequence SEQ ID NO: 22 and the TM and the first SC domain have together amino acid sequence SEQ ID NO: 24. According to some such embodiments, the leader peptide has the amino acid sequence SEQ ID NO: 13. According to one embodiment, the activation domain has amino acid sequence SEQ ID NO: 21. According to an alternative embodiment, the activation domain has amino acid sequence SEQ ID NO: 10. According to one embodiment, the present invention provides a nucleic acid construct comprising an isolated nucleic acid encoding a CAR having the sequence SEQ ID NO: 15. According to another embodiment, the CAR consists of the amino acid sequence SEQ ID NO: 15. According to a further embodiment, the present invention provides a nucleic acid construct comprises an isolated nucleic acid encoding a CAR having the amino acid sequence SEQ ID NO: 27. According to yet another embodiment, the CAR consists of the amino acid sequence SEQ ID NO: 27.

According to one embodiment, the nucleic acid construct comprises a nucleic acid selected from SEQ ID NO: 43, 44, 45, and 46. According to another embodiment, the nucleic acid construct comprises a nucleic acid consisting of nucleic acid sequence selected from SEQ ID NO: 43, 44, 45, and 46.

According to another aspect, the present invention provides a vector comprising the isolated nucleic acid of the present invention.

According to another embodiment, the present invention provides a vector comprising the nucleic acid construct of the present invention.

The terms “vector” and “expression vector” are used herein interchangeably and refer to any viral or non-viral vector such as plasmid, virus, retrovirus, bacteriophage, cosmid, artificial chromosome (bacterial or yeast), phage, binary vector in double or single stranded linear or circular form, or nucleic acid, sequence which is able to transform host cells and optionally capable of replicating in a host cell. The vector may be integrated into the cellular genome or may exist extrachromosomally (e.g., autonomous replicating plasmid with an origin of replication). The vector may contain an optional marker suitable for use in the identification of transformed cells. A cloning vector may or may not possess the features necessary for it to operate as an expression vector. The vector according to the invention is used for transferring the nucleic acid of the present invention to a host cell. The vector optionally comprises a viral capsid or other materials for facilitating entry of the nucleic acid into the host cell and/or replication of the vector in the host cell.

According to other embodiments, the vector is a virus, e.g. a modified or engineered virus. The modification of a vector may include mutations, such as deletion or insertion mutation, gene deletion or gene inclusion. In particular, a mutation may be done in one or more regions of the viral genome. Such mutations may be introduced in a region related to internal structural proteins, replication, or reverse transcription function. Other examples of vector modification are deletion of certain genes constituting the native infectious vector such as genes related to the virus' pathogenicity and/or to its ability to replicate.

According to some embodiments, the virus is a dsDNA virus (e.g. Adenoviruses, Herpesviruses, Poxviruses), a single stranded “plus” sense DNA virus (e.g., Parvoviruses) a double stranded RNA virus (e.g., Reoviruses), a single stranded+sense RNA virus (e.g. Picornaviruses, Togaviruses), a single stranded “minus” sense RNA virus (e.g. Orthomyxoviruses, Rhabdoviruses), a single stranded+sense RNA virus with a DNA intermediate (e.g. Retroviruses), or a double stranded reverse transcribing virus (e.g. Hepadnaviruses). In certain non-limiting embodiments of the present invention, the virus is poliovirus (PV), rhinovirus, influenza virus including avian flu (e.g. H5N1 subtype of influenza A virus), severe acute respiratory syndrome (SARS) coronavirus, Human Immunodeficiency Virus (HIV), Hepatitis B Virus (HBV), Hepatitis C Virus (HCV), infectious bronchitis virus, ebolavirus, Marburg virus, dengue fever virus (Flavivirus serotypes), West Nile disease virus, Epstein-Barr virus (EBV), yellow fever virus, Ebola (ebolavirus), chickenpox (varicella-zoster virus), measles (a paramyxovirus), mumps (a paramyxovirus), rabies (Lyssavirus), human papillomavirus, Kaposi's sarcoma-associated herpesvirus, Herpes Simplex Virus (HSV Type 1), or genital herpes (HSV Type 2).

According to any one of the above embodiments, the virus is a non-pathogenic virus or a modified virus lacking pathogenic genes.

According to some embodiments, the vector is a virus selected from lentivirus, adenovirus, modified adenovirus and retrovirus. In one particular embodiment, the vector is lentivirus.

Thus in one embodiment, the present invention provides a vector, comprising a nucleic acid encoding a CAR, wherein the CAR comprises in order from N-Terminus to C-Terminus:
(i) a leader peptide;
(ii) a Her2 binding domain comprising a light chain variable region (V_{L}) having amino acid sequence SEQ ID NO: 11 and a heavy chain variable region (V_{H}) having amino acid sequence SEQ ID NO: 12, wherein the V_{L} and the V_{H} are linked by a peptide linker;
(iii) a hinge region selected from the hinge region of CD28 and CD8,
(iv) a transmembrane (TM) domain selected from the TM domain of CD28 and ICOS;
(v) a first co-stimulatory (CS) domain selected from the co-stimulatory domain of CD28 and ICOS;
(vi) a second co-stimulatory domain which is the co-stimulatory domain of human 4-1BB receptor having amino acid sequence SEQ ID NO: 9; and
(vii) an activation domain selected from the human activation domain CD3ζ having the amino acid sequence SEQ ID NO: 21 and the activation domain of human Fcγ receptor (FcγR) having amino acid sequence SEQ ID NO: 10.

According to some such embodiments, the vector is virus. According to another embodiment, the vector is a lentivirus.

According to one embodiment, the vector of the present invention comprises a nucleic acid encoding a CAR of the present invention in which the Her2 binding domain has amino acid sequence SEQ ID NO: 23 and the activation domain has amino acid sequence SEQ ID NO: 21. According to one embodiment, the hinge region has amino acid sequence SEQ ID NO: 19. According to another embodiment, the leader peptide has amino acid sequence SEQ ID NO: 26. According to yet another embodiment, the hinge region has amino acid sequence SEQ ID NO: 19 and the leader peptide has amino acid sequence SEQ ID NO: 26. According to one such embodiment, the TM and first SC domain have together amino acid sequence SEQ ID NO: 24. According to another such embodiment, the TM and first SC domain have together amino acid sequence SEQ ID NO: 20. According to one embodiment, the present invention provides a vector comprising an isolated nucleic acid encoding a chimeric antigen receptor having the amino acid sequence SEQ ID NO: 28. According to another embodiment, the CAR consists of the amino acid sequence SEQ ID NO: 28. According to a further embodiment, the present invention provides a vector comprising an isolated nucleic acid encoding a chimeric antigen receptor having the amino acid sequence SEQ ID NO: 29. According to another embodiment, the CAR consists of the amino acid sequence SEQ ID NO: 29.

According to alternative embodiments, the vector of the present invention comprises a nucleic acid encoding a CAR of the present invention in which the Her2 binding domain has amino acid sequence SEQ ID NO: 23, the hinge region has amino acid sequence SEQ ID NO: 19 and the activation domain has amino acid sequence SEQ ID NO: 21. According to some embodiments, the leader peptide has amino acid sequence SEQ ID NO: 13.

According to one embodiment, the vector of the present invention comprises a nucleic acid encoding a CAR in which wherein Her2 binding domain has the amino acid sequence SEQ ID NO: 23, the hinge region has the amino acid sequence SEQ ID NO: 22 and the TM and the first SC domain have together amino acid sequence SEQ ID NO: 24. According to some such embodiments, the leader peptide has the amino acid sequence SEQ ID NO: 13. According to one embodiment, the activation domain has amino acid sequence SEQ ID NO: 21. According to an alternative embodiment, the activation domain has amino acid sequence SEQ ID NO: 10. According to one embodiment, the present invention provides a vector comprising an isolated nucleic acid encoding a CAR having the sequence SEQ ID NO: 15. According to another embodiment, the CAR consists of the amino acid sequence SEQ ID NO: 15. According to a further embodiment, the present invention provides a vector comprising an isolated nucleic acid encoding a CAR having the amino acid sequence SEQ ID NO: 27. According to yet another embodiment, the CAR consists of the amino acid sequence SEQ ID NO: 27.

According to one embodiment, the vector of the present invention comprises a nucleic acid having a nucleic acid sequence selected from SEQ ID NO: 43, 44, 45, and 46. According to another embodiment, the vector of the present invention comprises a nucleic acid consisting of a nucleic acid sequence selected from SEQ ID NO: 43, 44, 45, and 46. According to some such embodiments, the vector is virus. According to another embodiment, the vector is a lentivirus.

According to another aspect, the present invention provides a genetically modified T cell comprising the isolated nucleic acid of the present invention. According to another embodiment, the present invention provides a genetically modified T cell comprising the nucleic acid construct of the present invention. According to yet another embodiment, the present invention provides a genetically modified T cell comprising the vector of the present invention. The terms “genetically modified T cell” of the present invention and “CAR-T cell” are used herein interchangeably.

Thus, in some embodiments, the present invention provides a genetically modified T cell comprising a nucleic acid encoding a CAR, wherein the CAR comprises in order from N-Terminus to C-Terminus:
(i) a leader peptide;
(ii) a Her2 binding domain comprising a light chain variable region (V_{L}) having amino acid sequence SEQ ID NO: 11 and a heavy chain variable region (V_{H}) having amino acid sequence SEQ ID NO: 12, wherein the V_{L} and the V_{H} are linked by a peptide linker;
(iii) a hinge region selected from the hinge region of CD28 and CD8,
(iv) a transmembrane (TM) domain selected from the TM domain of CD28 and ICOS;
(v) a first co-stimulatory (CS) domain selected from the co-stimulatory domain of CD28 and ICOS;
(vi) a second co-stimulatory domain which is the co-stimulatory domain of human 4-1BB receptor having amino acid sequence SEQ ID NO: 9; and
(vii) an activation domain selected from the human activation domain CD3ζ having the amino acid sequence SEQ ID NO: 21 and the activation domain of human Fcγ receptor (FcγR) having amino acid sequence SEQ ID NO: 10.

According to one embodiment, the present invention provides a genetically modified T cell comprising a nucleic acid encoding a CAR in which the Her2 binding domain has amino acid sequence SEQ ID NO: 23 and the activation domain has amino acid sequence SEQ ID NO: 21. According to one embodiment, the hinge region has amino acid sequence SEQ ID NO: 19. According to another embodiment, the leader peptide has amino acid sequence SEQ ID NO: 26. According to yet another embodiment, the hinge region has amino acid sequence SEQ ID NO: 19 and the leader peptide has amino acid sequence SEQ ID NO: 26. According to one such embodiment, the TM and first SC domain have together amino acid sequence SEQ ID NO: 24. According to another such embodiment, the TM and first SC domain have together amino acid sequence SEQ ID NO: 20. According to one embodiment, the present invention provides a genetically modified T cell comprising a nucleic acid encoding a CAR having the amino acid sequence SEQ ID NO: 28. According to another embodiment, the CAR consists of the amino acid sequence SEQ ID NO: 28. According to a further embodiment, the present invention provides a genetically modified T cell comprising a nucleic acid encoding a CAR having the amino acid sequence SEQ ID NO: 29. According to another embodiment, the CAR consists of the amino acid sequence SEQ ID NO: 29.

According to alternative embodiments, the present invention provides a genetically modified T cell comprising a nucleic acid encoding a CAR in which the Her2 binding domain has amino acid sequence SEQ ID NO: 23, the hinge region has amino acid sequence SEQ ID NO: 19 and the activation domain has amino acid sequence SEQ ID NO: 21. According to some embodiments, the leader peptide has amino acid sequence SEQ ID NO: 13.

According to one embodiment, the present invention provides a genetically modified T cell comprising a nucleic acid encoding a CAR in which wherein Her2 binding domain has the amino acid sequence SEQ ID NO: 23, the hinge region has the amino acid sequence SEQ ID NO: 22 and the TM and the first SC domain have together amino acid sequence SEQ ID NO: 24. According to some such embodiments, the leader peptide has the amino acid sequence SEQ ID NO: 13. According to one embodiment, the activation domain has amino acid sequence SEQ ID NO: 21. According to an alternative embodiment, the activation domain has amino acid sequence SEQ ID NO: 10. According to one embodiment, the present invention provides a genetically modified T cell comprising a nucleic acid encoding a CAR having the sequence SEQ ID NO: 15. According to another embodiment, the CAR consists of the amino acid sequence SEQ ID NO: 15. According to a further embodiment, the present invention provides a genetically modified T cell comprising a nucleic acid encoding a CAR having the amino acid sequence SEQ ID NO: 27. According to yet another embodiment, the CAR consists of the amino acid sequence SEQ ID NO: 27.

According to one embodiment, the CAR-T cell comprises a nucleic acid having a nucleic acid sequence selected from SEQ ID NO: 43, 44, 45, and 46. According to another embodiment, the CAR-T cell comprises a nucleic acid consisting of a nucleic acid sequence selected from SEQ ID NO: 43, 44, 45, and 46.

According to one embodiment, the genetically modified T cell of the present invention is capable of expressing the CAR of the present invention. According to another embodiment, the genetically modified T cell of the present invention expresses the CAR of the present invention. According to one embodiment, the CAR is as described in any one of the above aspects and embodiments. According to one embodiment, the CAR-T cell expresses a CAR comprising an amino acid sequence selected from SEQ ID NO: 27, 28, 29 and 15. According to another embodiment, the CAR-T cell expresses a CAR consisting of an amino acid sequence selected from SEQ ID NO: 27, 28, 29 and 15. According to a further embodiment, the CAR-T cell is capable of expressing a CAR comprising or consisting of an amino acid sequence selected from SEQ ID NO: 27, 28, 29 and 15.

According to any one of the above embodiments, the T-cell is selected from cytotoxic lymphocyte cells, central memory T cells, effector memory T cells and memory stem cells. In some embodiments, such T cells are CD8+, CD3+ or CD4+ T cells.

According to some embodiments, the CAR-T cell of the present invention exhibits at least one of the following: higher cell surface expression, increased cytotoxicity, higher expansion, higher CAR-T cell proliferation upon activation, higher persistence, and lower exhaustion rate in comparison to known N29 CAR T-cells.

According to a further aspect, the present invention provides a population of T cells comprising a plurality of the genetically modified T cells according to the present invention. Thus, the present invention provides a population of T cells comprising a plurality of the genetically modified T cells, wherein the CAR-T cells comprise an isolated nucleic acid encoding a CAR, wherein the CAR comprises in order from N-Terminus to C-Terminus:
(i) a leader peptide;
(ii) a Her2 binding domain comprising a light chain variable region (V_{L}) having amino acid sequence SEQ ID NO: 11 and a heavy chain variable region (V_{H}) having amino acid sequence SEQ ID NO: 12, wherein the V_{L} and the V_{H} are linked by a peptide linker;
(iii) a hinge region selected from the hinge region of CD28 and CD8,
(iv) a transmembrane (TM) domain selected from the TM domain of CD28 and ICOS;
(v) a first co-stimulatory (CS) domain selected from the co-stimulatory domain of CD28 and ICOS;
(vi) a second co-stimulatory domain which is the co-stimulatory domain of human 4-1BB receptor having amino acid sequence SEQ ID NO: 9; and
(vii) an activation domain selected from the human activation domain CD3ζ having the amino acid sequence SEQ ID NO: 21 and the activation domain of human Fcγ receptor (FcγR) having amino acid sequence SEQ ID NO: 10.

According to one embodiment, the population of T cells comprises a plurality of CAR-T cells of the present invention which are capable of expressing the CAR of the present invention. According to another embodiment, the CAR-T cell of the present invention expresses the CAR of the present invention. According to one embodiment, the CAR is as described in any one of the above aspects and embodiments. According to one embodiment, the CAR-T cells express a CAR comprising an amino acid sequence selected from SEQ ID NO: 27, 28, 29 and 15. According to another embodiment, the CAR-T cells express a CAR consisting of an amino acid sequence selected from SEQ ID NO: 27, 28, 29 and 15. According to a further embodiment, the CAR-T cells are capable of expressing a CAR comprising or consisting of an amino acid sequence selected from SEQ ID NO: 27, 28, 29 and 15. According to any one of the above embodiments, the CAR T-cells are selected from cytotoxic lymphocyte cells, central memory T cells, effector memory T cells and memory stem cells. In some embodiments, such T cells are CD8+, CD3+ or CD4+T cells.

According to some embodiments, the T cells population of the present invention is an enriched population of CAR-T cells. According to one embodiment, the enriched population comprises at least 20% of CAR-T cells. According to another embodiment, the T-cells population comprises at least 25%, at least 30%, at least 35% or at least 40% genetically modified T cells of the present invention. According to another embodiment, the enriched population comprises at least 50%, at least 60%, or at least 70% genetically modified T cells of all T cells. According to one embodiment, the enriched T cells population comprises from about 20% to about 50%, from about 25% to about 45%, about 30% to about 40% of CAR-T cells of the present invention. According to a further embodiment, the enriched population comprises from about 40% to about 90%, about 45% to about 85%, about 50% to about 80%, about 55% to about 75%, or about 60% to about 70% genetically modified T cells. According to some embodiments, the enriched population comprises from about 70% to about 90%, from about 75% to about 85% genetically modified T cells of the present invention.

According to some embodiments, the T cells population is enriched using the marker present in the CAR. According to some embodiments, the enriched T cells population is obtained by a purification of the original T cell population using affinity chromatography.

According to one embodiment, the CAR-T cells of the population exhibit at least one of higher cell surface expression, increased cytotoxicity, higher CAR-T cell proliferation upon activation, higher persistence, and lower exhaustion rate in comparison to N29 CAR-T cells.

According to one embodiment, the CAR T-cells exhibit higher cell surface expression of the CAR. According to one embodiment, the higher cell surface expression of the CAR comprises from 10% to 100%, from 20% to 90%, from 30% to 80%, from 40% to 70% or from 50 to 60% higher cell surface expression than of known N29 CAR-T cells. According to another embodiment, the higher cell surface expression of the CAR comprises from 5% to 25%, from 25% to 50% from 50% to 75% or from 75% to 100% higher cell surface expression of the CAR. According to a further embodiment, the higher cell surface expression of the CAR comprises at least 1.5, at least 2, at least 2.5 at least 3, at least 5 or at least 10 folds higher cell surface expression of the CAR.

According to one embodiment, the higher proliferation comprises from 10% to 100%, from 20% to 90%, from 30% to 80%, from 40% to 70% or from 50% to 60% higher proliferation than of known N29 CAR-T cells. According to another embodiment, the higher proliferation comprises from 5% to 25%, from 25% to 50% from 50% to 75% or from 75% to 100% higher proliferation. According to a further embodiment, the higher proliferation comprises at least 1.5, at least 2, at least 2.5 at least 3, at least 5 or at least 10 folds higher proliferation.

The term “exhaustion” of T cells refers to a poor effector function and sustained expression of inhibitory receptors in comparison to T cells at t=0. According to one embodiment, the lower exhaustion rate comprises from 10% to 95%, from 20% to 90%, from 30% to 80%, from 40% to 70% or from 50% to 60% lower exhaustion rate than in N29 CAR-T cells. According to another embodiment, the lower exhaustion rate comprises from 5% to 25%, from 25% to 50% from 50% to 75% or from 75% to 95% lower exhaustion.

According to any one of the above embodiments, the population of T cells is administered to a subject in need thereof. According to some embodiments, the T cells of the present invention have an enhanced persistence in the serum. The term “persistence” refers to the presence of the CAR-T cells in the serum after intravenous (IV) administration. This terms correlates to the t0.5 of the cells in the blood stream. According to one embodiment, the higher persistence comprises from 10% to 100%, from 20% to 90%, from 30% to 80%, from 40% to 70% or from 50% to 60% higher persistence than of N29 CAR-T cells. According to another embodiment, the higher persistence comprises from 10% to 30% or from 15% to 20% higher persistence. According to a further embodiment, the higher persistence comprises more than 10, more than 15%, more than 20%, more than 25% or more than 30% higher persistence than the N29 CAR-T cells. According to some embodiments, said CAR-T cells are present in the serum at least 4 weeks after IV administration. According to some embodiments, said CAR-T cells are present in the serum at least 5, at least 6, at least 8 or at least 10 weeks after IV administration. According to certain embodiments, said CAR-T cells are present in the serum at least 12, at least 14, at least 16, at least 20, at least 24, or at least 30 weeks after IV administration. According to some other embodiments, the CAR T cells are present in the serum at least 3, at least 4, at least 5, at least 6, at least 7, at least 8, at least 9, at least 10, at least 11 or at least 12 months after IV administration. According to some other embodiments, the CAR T cells expressing the CAR of the present invention are present in the serum at least 1, 1.5, 2, 2.5, 3, 3.5, 4, 4.5 or 5 years after IV administration. According to some embodiments, the persistence refers to at least 20% of the administered cells. Thus, according to one embodiment, at least 20% of the administered CAR-T cells are present in the serum at least 4 weeks after IV administration. According to other embodiments, the persistence refers to at least 25%, at least 30%, at least 35%, at least 40% or at least 45% of the administered CAR-T cells. According to a further embodiment, the persistence refers to at least 50%, at least 60%, at least 70%, at least 80% or at least 90% of the administered CAR-T cells. Thus in some embodiments, at least 25%, at least 30%, or at least 40% of the administered CAR-T cells persist in the serum for at least 4, at least 5, at least 6, at least 8, at least 10 or at least 12 weeks after IV administration. According to another embodiment, at least 25%, at least 30%, or at least 40% of the administered CAR-T cells persist in the serum for at least 1, at least 2, at least 3, at least 4, at least 5, at least 6, at least 7, at least 8, at least 9, at least 10, at least 11 or at least 12 months. According so some embodiments, the CAR-T cells of the present invention persist for at least 14, 16, 18, 20, 22, 24, 26, 28, 30, 32, 34 or 36 months after IV administration.

According to some embodiments, the CAR T cells of the present invention provide an increased efficacy in comparison to the known CAR-T cells targeting Her2/neu oncogene, and in particular to CAR-T cells targeting the same epitope of Her2/neu oncogene as the CAR of the present invention. The efficacy may be tested by any known method such as cytotoxicity assays or cytokine secretion assays. According to some embodiments, the CAR-T cells of the present invention provide an increase of at least 20% or at least 30% or at least 40%, 50%, 60%, 70%, 80% or 90% in cytokine secretion and/or cytotoxicity in comparison to the known CAR-T cells targeting Her2/neu oncogene, and in particular to CAR-T cells targeting the same epitope of Her2/neu oncogene. According to another embodiment, the CAR-T cells of the present invention provide an increase of 2, 2.5, 3, 3.5, 4, 4.5 or 5 times in cytokine secretion and/or cytotoxicity. According to some embodiments, the increased cytokine secretion and/or cytotoxicity is in comparison to 2^{nd} generation CAR-T cells targeting the same epitope of Her2/neu oncogene. For the purpose of the present invention, the 2^{nd} generation CAR-T refers to a CAR having less than 3 cytoplasmic domains selected from stimulatory, costimulatory and activation domains C-Terminally to the transmembrane domain.

According to another aspect, the present invention provides a pharmaceutical composition comprising a CAR-T cell according the present invention and a pharmaceutically acceptable carrier. According to another embodiment, the pharmaceutical composition comprises a plurality of the CAR-T cell according the present invention. According to another embodiment, the pharmaceutical composition comprises a population of T cells comprising a plurality of CAR-T cells of the present invention.

The term “pharmaceutical composition” as used herein refers to a composition comprising CAR-T cells of the present invention as disclosed herein optionally formulated together with one or more pharmaceutically acceptable carriers.

Formulation of the pharmaceutical composition may be adjusted according to applications. In particular, the pharmaceutical composition may be formulated using a method known in the art so as to provide rapid, continuous or delayed release of the active ingredient after administration to mammals. For example, the formulation may be any one selected from liquids, solutions, aerosols, emulsions, suspensions, infusions, ophthalmic solutions, tablets, injections, spirits, capsules, pills, and soft or hard gelatin capsules. According to some embodiments, the composition is formulated as an injectable composition, e.g. an injectable solution or suspension.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art. The compositions may contain other active compounds providing supplemental, additional, or enhanced therapeutic functions.

According to some embodiments, the present invention provides a pharmaceutical composition comprising a pharmaceutically acceptable carrier and a plurality of genetically modified T cells, wherein the genetically modified T cells comprise an isolated nucleic acid encoding a CAR, wherein the CAR comprises in order from N-Terminus to C-Terminus:
(i) a leader peptide;
(ii) a Her2 binding domain comprising a light chain variable region (V_{L}) having amino acid sequence SEQ ID NO: 11 and a heavy chain variable region (V_{H}) having amino acid sequence SEQ ID NO: 12, wherein the V_{L} and the V_{H} are linked by a peptide linker;
(iii) a hinge region selected from the hinge region of CD28 and CD8,
(iv) a transmembrane (TM) domain selected from the TM domain of CD28 and ICOS;
(v) a first co-stimulatory (CS) domain selected from the co-stimulatory domain of CD28 and ICOS;
(vi) a second co-stimulatory domain which is the co-stimulatory domain of human 4-1BB receptor having amino acid sequence SEQ ID NO: 9; and
(vii) an activation domain selected from the human activation domain CD3ζ having the amino acid sequence SEQ ID NO: 21 and the activation domain of human Fcγ receptor (FcγR) having amino acid sequence SEQ ID NO: 10.

According to one embodiment, the pharmaceutical composition of the present invention comprises a plurality CAR-T cells of the present invention expressing the CAR of the present invention. According to one embodiment, the CAR is as described in any one of the above aspects and embodiments. According to one embodiment, the CAR-T cells express a CAR comprising an amino acid sequence selected from SEQ ID NO: 27, 28, 29 and 15. According to another embodiment, the CAR-T cells express a CAR consisting of an amino acid sequence selected from SEQ ID NO: 27, 28, 29 and 15. According to one embodiment, the pharmaceutical composition of the present invention comprises a plurality CAR-T cells of the present invention capable of expressing a CAR comprising or consisting of an amino acid sequence selected from SEQ ID NO: 27, 28, 29 and 15. According to any one of the above embodiments, the CAR T-cells are selected from cytotoxic lymphocyte cells, central memory T cells, effector memory T cells and memory stem cells. In some embodiments, such T cells are CD8+, CD3+ or CD4+ T cells.

According to any one of the above embodiment, the pharmaceutical composition of the present invention is for use in treating cancer. According to some embodiments, the cancer is any cancer overexpressing Her2/neu oncogene. Non-limiting examples of such types of cancer are breast cancer, ovarian cancer, gastric cancer, glioblastoma, osteosarcoma, pancreatic cancer, lung non-small-cell carcinoma, head and neck cancer, prostate cancer and medulloblastoma. In one embodiment, cancer is breast cancer. According to one embodiment, the breast cancer is Her2 positive cancer. According to one embodiment, the breast cancer has a score of 3+ as tested by Her2 immunohistochemistry test. According to another embodiment, the breast cancer is borderline Her2 expressing cancer. According to one embodiment, the breast cancer has a score of 2+ as tested by Her2 immunohistochemistry test. According to yet another embodiment, the breast cancer has a score of 1+ as tested by Her2 immunohistochemistry test. The overexpression of the Her2 oncogene may be assessed by any known method and correlated to Her2 immunohistochemistry test.

The pharmaceutical composition of the present invention may be administered by any known method.

The terms “administering” or “administration of” a substance, a compound, a pharmaceutical composition or an agent, collectively called an entity, to a subject can be carried out using one of a variety of methods known to those skilled in the art. For example, the entity may be administered intravenously, arterially, intradermally, intramuscularly, intraperitoneally, intravenously, subcutaneously, ocularly, sublingually, orally (by ingestion), intranasally (by inhalation), intraspinally, intracerebrally, and transdermally (by absorption, e.g., through a skin duct). The entity can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

According to some embodiments, administering comprises administering via injection e.g. intramuscularly, intravenously (IV), intra-ocularly, subcutaneously, intradermally or transdermally injection. According to one embodiment, the administration is IV administration.

According to another aspect, the present invention provides a method of treating cancer in a subject in need thereof, comprising administering a therapeutically effective amount of the CAR-T cells according to the present invention.

The term “therapeutically effective amount” refers to an amount of a drug or an agent that, when administered to a subject will have the intended therapeutic effect, e.g. treatment of cancer. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of the cognitive impairment, and the therapeutics or combination of therapeutics selected for administration, and the mode of administration. The skilled worker can readily determine the effective amount for a given situation by routine experimentation. The term “treating” a disease, a condition or patient refers to taking steps to obtain beneficial or desired results, including clinical results. Beneficial or desired clinical results include, but are not limited to, or ameliorating abrogating, substantially inhibiting, slowing or reversing the progression of a disease, condition or disorder, substantially ameliorating or alleviating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and/or (e) limiting recurrence of symptoms in patients that were previously asymptomatic for the disorder(s).

According to some embodiments, the method comprises administering the pharmaceutical composition of the present invention comprising the CAR-T cells of the present invention. According to some embodiments, the method comprises administering a therapeutically effective amount of the CAR-T cells expressing or capable of expressing the CAR of the present invention such as CAR having the sequence selected from the group consisting of SEQ ID NO: 27, 28, 29 and 15. According to some embodiments, the cancer is any cancer overexpressing Her2/neu oncogene. According to some embodiments, the cancer is selected from the groups consisting of breast cancer, ovary cancer, gastric cancer, glioblastoma, osteosarcoma, and medulloblastoma. According to one embodiment, the cancer is breast cancer. According to one embodiment, the breast cancer has a score selected from 1+, 2+ and 3+ as tested by Her2 immunohistochemistry test. According to some embodiments, at least 20% of the administered said CAR-T cells are present in the serum at least 4 weeks after administration.

According to another aspect, the present invention provides use of CAR-T cells of the present invention in preparation of a medicament for treating cancer. According to one embodiment, the cancer is selected from breast cancer, ovary cancer, gastric cancer, glioblastoma, osteosarcoma, and medulloblastoma. According to another embodiment, the cancer is breast cancer. According to one embodiment, the cancer is a HER2 positive cancer, e.g. cancer having a score of 2+ or 3+ in a Her2 immunohistochemistry test. According to another embodiment, the breast cancer has a score of 1+ in Her2 immunohistochemistry test.

As used herein the terms “comprise(s)” “include(s),” “having,” “has,” “contain(s),” and variants thereof, are intended to be open-ended transitional phrases, terms, or words that do not preclude the possibility of additional acts or structure. Moreover, the term “comprising” always includes the term “consisting” and when the term “comprising” appears in an embodiment of the disclosure, this same embodiment wherein the term “consisting” replaces the term “comprising” is always also an embodiment of the disclosure.

As used herein, the term “about”, when referring to a measurable value such as an amount, a temporal duration, and the like, is meant to encompass variations of +/−10%, or +/−5%, +/−1%, or even +/−0.1% from the specified value.

Having now generally described the invention, the same will be more readily understood through reference to the following examples, which are provided by way of illustration and are not intended to be limiting of the present invention.

### EXAMPLES

### Example 1

### Design of N29 3^{rd} Generation CARs

Several 3^{rd} generation CARs based on N29 2^{nd} generation CAR are designed and prepared. These CARs improve the overall performance of this chimeric receptor compared to its current 2^{nd} generation counterpart.

The general structure of 3^{rd} generation anti-HER2 CAR is as follows:

N′—leader peptide—N29 recognition domain—hinge region—transmembrane domain—1^{st} co-stimulatory domain—2^{nd} co-stimulatory domain—activation domain—C′. The N29 recognition domain comprises V_{L} and V_{H} domains connected by a peptide linker.

The following CARs are designed and prepared; the relevant sequences are summarized in Table 1:

CAR-O: Igκ signal peptide—N29-V_{L}—peptide linker—N29-V_{H}—hinge region of CD28—TM domain of CD28—co-stimulatory (CS) domain of CD28—co-stimulatory domain of human 4-1BB—activation domain of human Fcγ receptor.

CAR-A: Igκ signal peptide—N29-V_{L}—peptide linker—N29-V_{H}—hinge region of CD28—TM domain of CD28—co-stimulatory domain of CD28—co-stimulatory domain of human 4-1BB—activation domain CD3ζ.

CAR-B: CD8α signal peptide—N29-V_{L}—peptide linker—N29-V_{H}—hinge region of CD8—TM domain of CD28—co-stimulatory domain of CD28—co-stimulatory domain of human 4-1BB—activation domain CD3ζ.

CAR-C: CD8α signal peptide—N29-V_{L}—peptide linker—N29-V_{H}—hinge region of CD8—TM of ICOS—co-stimulatory domain of ICOS—co-stimulatory domain of human 4-1BB—activation domain CD3ζ.

TABLE 1

| | |
|---|---|
| Igκ signal peptide | SEQ ID NO: 13 |
| N29-VL | SEQ ID NO: 11 |
| linker | SEQ ID NO: 14 |
| N29-VH | SEQ ID NO: 12 |
| CD28 hinge | SEQ ID NO: 22 |
| TM and CS domain of CD28 | SEQ ID NO: 24 |
| CS domain of 4-1BB | SEQ ID NO: 9 |
| activation domain CD3ζ | SEQ ID NO: 21 |
| activation domain of human Fcγ receptor | SEQ ID NO: 10 |
| CD8α signal peptide | SEQ ID NO: 26 |
| CD8α hinge | SEQ ID NO: 19 |
| TM + CS of ICOS | SEQ ID NO: 20 |
| CAR-O complete sequence | SEQ ID NO: 15 |
| CAR-A complete sequence | SEQ ID NO: 27 |
| CAR-B complete sequence | SEQ ID NO: 28 |
| CAR-C complete sequence | SEQ ID NO: 29 |

The sequences encoding the 4 prepared CARs are presented in Table 2.

TABLE 2
nucleic acid encoding the designed CARs

| | |
|---|---|
| CAR-O | ATGGATTTTCAGGTGCAGATTTTCAGCTTCCTGCTAATCAGTGC |
| | CTCAGTCATAATGTCTAGAGGAGATATTGTGATGACCCAGTCT |
| | CCAAAATTCATGTCCACATCAGTAGGAGACAGGATCAGCGTCA |
| | CCTGCAAGGCCAGTCAAGATGTGGGTCCTAATGTAGCCTGGTA |
| | TCAACAGAAACCAGGGCAATCTCCTAAACCACTGATTTACTCG |
| | GCATCCTACCTATATAATGGAGTCCCTGATCGCTTCACAGGCA |
| | GTGGATCTGGGACAGATTTCTCTCTCACCATCAGCAATGTGCA |
| | GTCTGATGACTTGGCAGAGTATTTCTGTCAGCAATATAACACC |
| | TATCCGTTCACGTTCGGAGGGGGCACCAAGCTGGAAATCAAA |
| | GGGTCGACTTCCGGTAGCGGCAAATCCTCTGAAGGCAAAGGT |
| | GAGGTGCAGCTGGAGGAGTCTGGTGGAGGATTGGTGCAGCCT |
| | AAAGGGTCATTGAAACTCTCATGTGCAGCCTCTGGATTCACCT |
| | TCAATACCTACGCCATGAACTGGGTCCGCCAGGCTCCAGGAAA |
| | GGGTTTGGAATGGATTGTTCGCATAAGAAGTAAAAGTAATAAT |
| | TATGCAACATATTATGTCGATTCAGTGAAAGACAGGTTCACCA |
| | TCTCCAGAGATGATTCACAAAGCATGCTCTATCTGCAAATGAA |
| | CAACTTGAAAACTGAGGACACAGCCATGTATTACTGTGTGACT |
| | TCTTACTATGATTACGACAAGGTCCTGTTTGCTTACTGGGGCCA |
| | AGGGACCACGGTCACCGTGAAAGGGAAACACCTTTGTCCAAG |
| | TCCCCTATTTCCCGGACCTTCTAAGCCCTTTTGGGTGCTGGTGG |
| | TGGTTGGTGGAGTCCTGGCTTGCTATAGCTTGCTAGTAACAGT |
| | GGCCTTTATTATTTTCTGGGTGAGGAGTAAGAGGAGCAGGCTC |
| | CTGCACAGTGACTACATGAACATGACTCCCCGCCGCCCCGGGC |
| | CCACCCGCAAGCATTACCAGCCCTATGCCCCACCACGCGACTT |
| | CGCAGCCTATAGATCTCGTTTCTCTGTTGTTAAACGGGGCAGA |
| | AAGAAGCTCCTGTATATATTCAAACAACCATTTATGAGACCAG |
| | TACAAACTACTCAAGAGGAAGATGGCTGTAGCTGCCGATTTCC |
| | AGAAGAAGAAGAAGGAGGATGTGAACTGCAAGTGCGAAAGG |
| | CAGCTATAACCAGCTATGAGAAATCAGATGGTGTTTACACGGG |
| | CCTGAGCACCAGGAACCAGGAGACTTACGAGACTCTGAAGCA |
| | TGAGAAACCACCACAGTAG |
| CAR-A | ATGGCCTTACCAGTGACCGCCTTGCTCCTGCCGCTGGCCTTGCT |
| | GCTCCACGCCGCCAGGCCGGATATTGTGATGACCCAGTCTCCA |
| | AAATTCATGTCCACATCAGTAGGAGACAGGATCAGCGTCACCT |
| | GCAAGGCCAGTCAAGATGTGGGTCCTAATGTAGCCTGGTATCA |
| | ACAGAAACCAGGGCAATCTCCTAAACCACTGATTTACTCGGCA |
| | TCCTACCTATATAATGGAGTCCCTGATCGCTTCACAGGCAGTG |
| | GATCTGGGACAGATTTCTCTCTCACCATCAGCAATGTGCAGTC |
| | TGATGACTTGGCAGAGTATTTCTGTCAGCAATATAACACCTAT |
| | CCGTTCACGTTCGGAGGGGGCACCAAGCTGGAAATCAAAGGG |
| | TCGACTTCCGGTAGCGGCAAATCCTCTGAAGGCAAAGGTGAG |
| | GTGCAGCTGGAGGAGTCTGGTGGAGGATTGGTGCAGCCTAAA |
| | GGGTCATTGAAACTCTCATGTGCAGCCTCTGGATTCACCTTCA |
| | ATACCTACGCCATGAACTGGGTCCGCCAGGCTCCAGGAAAGG |
| | GTTTGGAATGGATTGTTCGCATAAGAAGTAAAAGTAATAATTA |
| | TGCAACATATTATGTCGATTCAGTGAAAGACAGGTTCACCATC |
| | TCCAGAGATGATTCACAAAGCATGCTCTATCTGCAAATGAACA |
| | ACTTGAAAACTGAGGACACAGCCATGTATTACTGTGTGACTTC |
| | TTACTATGATTACGACAAGGTCCTGTTTGCTTACTGGGGCCAA |
| | GGGACCACGGTCACCGCAAAGCCAACAACCACTCCAGCCCCC |
| | CGGCCTCCAACGCCTGCGCCAACCATTGCATCACAACCGCTCA |
| | GCCTGAGACCCGAAGCGTGCAGACCCGCCGCAGGGGGAGCGG |
| | TCCATACACGGGGGCTGGACTTTGCTTGTGACTTTTGGGTGCT |
| | GGTGGTGGTTGGTGGAGTCCTGGCTTGCTATAGCTTGCTAGTA |
| | ACAGTGGCCTTTATTATTTTCTGGGTGAGGAGTAAGAGGAGCA |
| | GGCTCCTGCACAGTGACTACATGAACATGACTCCCCGCCGCCC |
| | CGGGCCCACCCGCAAGCATTACCAGCCCTATGCCCCACCACGC |
| | GACTTCGCAGCCTATAGATCTCGTTTCTCTGTTGTTAAACGGGG |
| | CAGAAAGAAGCTCCTGTATATATTCAAACAACCATTTATGAGA |
| | CCAGTACAAACTACTCAAGAGGAAGATGGCTGTAGCTGCCGA |
| | TTTCCAGAAGAAGAAGAAGGAGGATGTGAACTGAGAGTGAAG |
| | TTCAGCAGGAGCGCAGACGCCCCCGCGTACCAGCAGGGCCAG |
| | AACCAGCTCTATAACGAGCTCAATCTAGGACGAAGAGAGGAG |
| | TACGATGTTTTGGACAAGAGACGTGGCCGGGACCCTGAGATG |
| | GGGGGAAAGCCGCAGAGAAGGAAGAACCCTCAGGAAGGCCT |
| | GTACAATGAACTGCAGAAAGATAAGATGGCGGAGGCCTACAG |
| | TGAGATTGGGATGAAAGGCGAGCGCCGGAGGGGCAAGGGGCA |
| | CGATGGCCTTTACCAGGGTCTCAGTACAGCCACCAAGGACACC |
| | TACGACGCCCTTCACATGCAGGCCCTGCCCCCTCGCTAG |
| CAR-B | ATGGCCTTACCAGTGACCGCCTTGCTCCTGCCGCTGGCCTTGCT |
| | GCTCCACGCCGCCAGGCCGGATATTGTGATGACCCAGTCTCCA |
| | AAATTCATGTCCACATCAGTAGGAGACAGGATCAGCGTCACCT |
| | GCAAGGCCAGTCAAGATGTGGGTCCTAATGTAGCCTGGTATCA |
| | ACAGAAACCAGGGCAATCTCCTAAACCACTGATTTACTCGGCA |
| | TCCTACCTATATAATGGAGTCCCTGATCGCTTCACAGGCAGTG |
| | GATCTGGGACAGATTTCTCTCTCACCATCAGCAATGTGCAGTC |
| | TGATGACTTGGCAGAGTATTTCTGTCAGCAATATAACACCTAT |
| | CCGTTCACGTTCGGAGGGGGCACCAAGCTGGAAATCAAAGGG |
| | TCGACTTCCGGTAGCGGCAAATCCTCTGAAGGCAAAGGTGAG |
| | GTGCAGCTGGAGGAGTCTGGTGGAGGATTGGTGCAGCCTAAA |
| | GGGTCATTGAAACTCTCATGTGCAGCCTCTGGATTCACCTTCA |
| | ATACCTACGCCATGAACTGGGTCCGCCAGGCTCCAGGAAAGG |
| | GTTTGGAATGGATTGTTCGCATAAGAAGTAAAAGTAATAATTA |
| | TGCAACATATTATGTCGATTCAGTGAAAGACAGGTTCACCATC |
| | TCCAGAGATGATTCACAAAGCATGCTCTATCTGCAAATGAACA |
| | ACTTGAAAACTGAGGACACAGCCATGTATTACTGTGTGACTTC |
| | TTACTATGATTACGACAAGGTCCTGTTTGCTTACTGGGGCCAA |
| | GGGACCACGGTCACCGCAAAGCCAACAACCACTCCAGCCCCC |
| | CGGCCTCCAACGCCTGCGCCAACCATTGCATCACAACCGCTCA |
| | GCCTGAGACCCGAAGCGTGCAGACCCGCCGCAGGGGGAGCGG |
| | TCCATACACGGGGGCTGGACTTTGCTTGTGACTTTTGGGTGCT |
| | GGTGGTGGTTGGTGGAGTCCTGGCTTGCTATAGCTTGCTAGTA |
| | ACAGTGGCCTTTATTATTTTCTGGGTGAGGAGTAAGAGGAGCA |
| | GGCTCCTGCACAGTGACTACATGAACATGACTCCCCGCCGCCC |
| | CGGGCCCACCCGCAAGCATTACCAGCCCTATGCCCCACCACGC |
| | GACTTCGCAGCCTATAGATCTCGTTTCTCTGTTGTTAAACGGGG |
| | CAGAAAGAAGCTCCTGTATATATTCAAACAACCATTTATGAGA |
| | CCAGTACAAACTACTCAAGAGGAAGATGGCTGTAGCTGCCGA |
| | TTTCCAGAAGAAGAAGAAGGAGGATGTGAACTGAGAGTGAAG |
| | TTCAGCAGGAGCGCAGACGCCCCCGCGTACCAGCAGGGCCAG |
| | AACCAGCTCTATAACGAGCTCAATCTAGGACGAAGAGAGGAG |
| | TACGATGTTTTGGACAAGAGACGTGGCCGGGACCCTGAGATG |
| | GGGGGAAAGCCGCAGAGAAGGAAGAACCCTCAGGAAGGCCT |
| | GTACAATGAACTGCAGAAAGATAAGATGGCGGAGGCCTACAG |
| | TGAGATTGGGATGAAAGGCGAGCGCCGGAGGGGCAAGGGGCA |
| | CGATGGCCTTTACCAGGGTCTCAGTACAGCCACCAAGGACACC |
| | TACGACGCCCTTCACATGCAGGCCCTGCCCCCTCGCTAG |
| CAR-C | ATGGCCTTACCAGTGACCGCCTTGCTCCTGCCGCTGGCCTTGCT |
| | GCTCCACGCCGCCAGGCCGGATATTGTGATGACCCAGTCTCCA |
| | AAATTCATGTCCACATCAGTAGGAGACAGGATCAGCGTCACCT |
| | GCAAGGCCAGTCAAGATGTGGGTCCTAATGTAGCCTGGTATCA |
| | ACAGAAACCAGGGCAATCTCCTAAACCACTGATTTACTCGGCA |
| | TCCTACCTATATAATGGAGTCCCTGATCGCTTCACAGGCAGTG |
| | GATCTGGGACAGATTTCTCTCTCACCATCAGCAATGTGCAGTC |
| | TGATGACTTGGCAGAGTATTTCTGTCAGCAATATAACACCTAT |
| | CCGTTCACGTTCGGAGGGGGCACCAAGCTGGAAATCAAAGGG |
| | TCGACTTCCGGTAGCGGCAAATCCTCTGAAGGCAAAGGTGAG |
| | GTGCAGCTGGAGGAGTCTGGTGGAGGATTGGTGCAGCCTAAA |
| | GGGTCATTGAAACTCTCATGTGCAGCCTCTGGATTCACCTTCA |
| | ATACCTACGCCATGAACTGGGTCCGCCAGGCTCCAGGAAAGG |
| | GTTTGGAATGGATTGTTCGCATAAGAAGTAAAAGTAATAATTA |
| | TGCAACATATTATGTCGATTCAGTGAAAGACAGGTTCACCATC |
| | TCCAGAGATGATTCACAAAGCATGCTCTATCTGCAAATGAACA |
| | ACTTGAAAACTGAGGACACAGCCATGTATTACTGTGTGACTTC |
| | TTACTATGATTACGACAAGGTCCTGTTTGCTTACTGGGGCCAA |
| | GGGACCACGGTCACCGCAAAGCCAACAACCACTCCAGCCCCC |
| | CGGCCTCCAACGCCTGCGCCAACCATTGCATCACAACCGCTCA |
| | GCCTGAGACCCGAAGCGTGCAGACCCGCCGCAGGGGGAGCGG |
| | TCCATACACGGGGGCTGGACTTTGCTTGTGACTTTTGGCTTCCC |
| | ATTGGTTGCGCGGCGTTTGTCGTAGTCTGCATCCTGGGATGCA |
| | TTCTCATTTGTTGGTTGACGAAGAAGAAATATAGTTCCTCAGT |
| | GCATGATCCAAACGGTGAATACATGTTTATGAGGGCCGTAAAC |
| | ACAGCTAAGAAGTCTCGCTTGACTGACGTTACACTGCGTTTCT |
| | CTGTTGTTAAACGGGGCAGAAAGAAGCTCCTGTATATATTCAA |
| | ACAACCATTTATGAGACCAGTACAAACTACTCAAGAGGAAGA |
| | TGGCTGTAGCTGCCGATTTCCAGAAGAAGAAGAAGGAGGATG |
| | TGAACTGAGAGTGAAGTTCAGCAGGAGCGCAGACGCCCCCGC |
| | GTACCAGCAGGGCCAGAACCAGCTCTATAACGAGCTCAATCTA |
| | GGACGAAGAGAGGAGTACGATGTTTTGGACAAGAGACGTGGC |
| | CGGGACCCTGAGATGGGGGGAAAGCCGCAGAGAAGGAAGAA |
| | CCCTCAGGAAGGCCTGTACAATGAACTGCAGAAAGATAAGAT |
| | GGCGGAGGCCTACAGTGAGATTGGGATGAAAGGCGAGCGCCG |
| | GAGGGGCAAGGGGCACGATGGCCTTTACCAGGGTCTCAGTAC |
| | AGCCACCAAGGACACCTACGACGCCCTTCACATGCAGGCCCTG |
| | CCCCCTCGCTAG |

### Example 2

### Packaging Cell Line Generation

Phoenix-GP cells (HEK 293T stably expressing γ-retroviral gag and pol proteins) are transfected with VSV-G and a γ-retroviral vector (MSGV1) containing the constructs prepared in Example 1 and encoding the CARs of the present invention and the control CAR (2^{nd} generation N29 CAR). These transfected cells produce viral particles used to infect PG-13 cells (3T3 cells stably expressing γ-retroviral gag, pol, and GalV env proteins). Viral particles secreted by infected PG-13 cells are collected and used to infect PBMCs.

### Example 3

### Transfection of CAR to PBMC

Human PBMCs (Peripheral Blood Mononuclear Cells) from enriched leukocyte blood units are taken from healthy donors. Isolation is performed by Ficoll density gradient centrifugation. The isolated PBMCs are activated by incubation with αCD3-αCD28 activating antibodies for 48 hours. The activated PBMCs are virally transduced by spinoculation with viral particles produced by infected PG-13 cells obtained in Example 2.

### Example 4

### Assessment of CARs Efficiency

48-72 hours post infection, the transduced PBMCs (“effector cells”) are co-cultured with target cells that express Her2—e.g. breast cancer cell lines BT474 and AU565. As a negative control, the transduced PBMCs are incubated with a non-tumorigenic breast cell line that does not express Her2—MCF10.

After 24 hours of co-culture, the culture media is collected and analyzed for different cytokine levels such as IL-2, IFN-γ, TNF-α—by ELISA. We also collect the PBMCs and stain for activation markers such as CD69, CD25, 4-1BB, OX-40 and CD107a (degranulation). Exhaustion markers—TIM3, LAG1, PD-1 are checked as well. These are analyzed by flow cytometry. The activatory cytokine levels and activation marker staining are shown to be higher in PBMCs transduced with the CARs of the present invention, than in the ones transduced with the control CAR (2^{nd} generation CAR). In addition the exhaustion markers are lower in PBMCs transduced with the proposed CAR than in ones transduced with the control CAR.

After 48-72 hours of co-culture, killing of target cells is assessed by different assays. The amount of killing is higher in PBMCs transduced with the CAR of the present invention, than in the ones transduced with the control CAR.

The following functional assays are performed:

### Expansion and Survival

The proliferative potential of N29 (2^{nd} generation), the CARs of the present invention (3^{rd} generation) and anti-TNP (control) CAR-transduced T cells is assessed by incubating them with Her2⁺ and Her2⁻ cancer cell lines and analyzing CAR-transduced cells by FACS at different time points for at least 20 days.

### Cytokines Release Assay

The assay is carried out by an ELISA Kit. Antigen-specific cytokines secretion by N29, the CARs of the present invention and anti-TNP (control) CAR-transduced T cells is tested on the supernatants following overnight incubation with Her2⁺ and Her2⁻ cancer cell lines.

### Cytotoxicity Assays

CD107a degranulation assays is conducted by co-incubating N29, the CARs of the present invention and anti-TNP (control) CAR-transduced T cells with Her2⁺ and Her2⁻ cancer cell lines for 24 hours in the presence of an anti-CD107a antibody and then measuring the levels of CD107a in the surface of the transduced T-cells by FACS analysis.

Killing assays are carried out by Crystal Violet and Calcein AM assays, after 48 h or 4-8 h respectively, of co-incubation of N29, the CARs of the present invention and anti-TNP (control) CAR-transduced T cells with Her2⁺ and Her2⁻ cancer cell lines.

### Differentiation

To test the differentiation status of N29, the CARs of the present invention and anti-TNP (control) CAR-transduced T cells, we use a standard panel of cell-surface markers associated with T cell differentiation. We assess expression of CD45RO and CCR7, which are associated with central memory (TCM) and effector memory (TEM) cells. T cells transduced with TNP-CAR, N29-CAR and the CARs of the present invention are plated at different E:T ratios. Following 7-14 days of incubation at 37° C., T cells are collected and assessed by FACS analysis.

### 5) Exhaustion

This phenotype is evaluated via FACS analysis of expression of PD-1, TIM-3 and LAG-3 proteins on T cells transduced with N29-CAR, the CARs of the present invention and anti-TNP (control) at different time points after co-culture with Her2⁺ and Her2⁻ cancer cell lines.

Although the present invention has been described herein above by way of preferred embodiments thereof, it can be modified, without departing from the spirit and nature of the subject invention as defined in the appended claims.

1-43. (canceled)

44. An isolated nucleic acid encoding a chimeric antigen receptor (CAR), wherein the CAR comprises in order from N-Terminus to C-Terminus:
(i) a leader peptide;
(ii) a Her2 binding domain comprising a light chain variable region (VL) having amino acid sequence SEQ ID NO: 11 and a heavy chain variable region (VH) having amino acid sequence SEQ ID NO: 12, wherein the VL and the VH are linked by a peptide linker;
(iii) a hinge region selected from the hinge region of CD28 and CD8,
(iv) a transmembrane (TM) domain selected from the TM domain of CD28 and ICOS;
(v) a first co-stimulatory (CS) domain selected from the co-stimulatory domain of CD28 and ICOS;
(vi) a second co-stimulatory domain which is the co-stimulatory domain of human 4-1BB receptor having amino acid sequence SEQ ID NO: 9; and
(vii) an activation domain selected from the human activation domain CD3ζ having amino acid sequence SEQ ID NO: 21 and the activation domain of human Fcγ receptor (FcγR) having amino acid sequence SEQ ID NO: 10.

45. The isolated nucleic acid according to claim 44, characterized by at least one of:
(i) the leader peptide has an amino acid sequence selected from SEQ ID NO: 13 and SEQ ID NO: 26;
(ii) the peptide linker consists of 10-30 amino acids;
(iii) the TM domain and the first SC domain are the TM domain and the CS domain of CD28 or wherein the TM domain and the SC domain are the TM domain and the CS domain of ICOS;
(iv) the activation domain has an amino acid sequence SEQ ID NO: 21;
(v) the hinge region of CD28 has amino acid sequence SEQ ID NO: 22 and the hinge region of CD8 has amino acid sequence SEQ ID NO: 19; and
(vi) the Her2 binding domain is a single-chain variable fragment (ScFv).

46. The isolated nucleic acid according to claim 45, characterized by at least one of:
(i) the peptide linker has amino acid sequence SEQ ID NO: 14;
(ii) the TM domain and the first SC domain together have an amino acid sequence selected from SEQ ID NO: 24 and SEQ ID NO: 20; and
(iii) the Her2 binding domain has amino acid sequence SEQ ID NO: 23.

47. The isolated nucleic acid of claim 44, wherein the CAR further comprises a co-stimulatory domain selected from a CS domain of CD80, OX40, CD154, CD27, and CD244.

48. The isolated nucleic acid according to claim 44, wherein the Her2 binding domain has amino acid sequence SEQ ID NO: 23 and the activation domain has amino acid sequence SEQ ID NO: 21.

49. The isolated nucleic acid according to claim 48, characterized by at least one of:
(i) the hinge region has amino acid sequence SEQ ID NO: 19;
(ii) the leader peptide has amino acid sequence SEQ ID NO: 26; and
(iii) the TM and first SC domain have together an amino acid sequence selected from SEQ ID NO: 24 and SEQ ID NO: 20.

50. The isolated nucleic acid according to claim 49, wherein the CAR comprises an amino acid sequence selected from SEQ ID NO: 28 and SEQ ID NO: 29.

51. The isolated nucleic acid according to claim 44, wherein the Her2 binding domain has amino acid sequence SEQ ID NO: 23, the hinge region has the amino acid sequence SEQ ID NO: 22 and the TM and the first SC domain have together amino acid sequence SEQ ID NO: 24.

52. The isolated nucleic acid according to claim 44, wherein the CAR comprises an amino acid sequence selected from SEQ ID NO: 15, 27, 43, 44, 45 and 46.

53. A nucleic acid construct comprising the nucleic acid of claim 44, operably linked to a promoter.

54. A vector comprising the isolated nucleic acid of claim 44.

55. A genetically modified T cell (CAR-T cell) comprising the isolated nucleic acid of claim 44 or a vector comprising the isolated nucleic acid of claim 44.

56. The genetically modified T cell according to claim 55, expressing the CAR encoded by said isolated nucleic acid.

57. A population of T cells comprising a plurality of the genetically modified CAR T cells according to claim 55.

58. The population according to claim 57, characterized by at least one of:
(i) the population is an enriched population comprising at least 30% genetically modified T cells;
(ii) the T cells exhibit at least one of higher cell surface expression, increased cytotoxicity, higher CAR-T cell proliferation upon activation, higher persistence, and lower exhaustion rate in comparison to N29 CAR T-cells; and
(iii) at least 20% of the administered said T cells upon intravenous administration persist in the serum at least 4 weeks after administration.

59. A pharmaceutical composition comprising the genetically modified T cells according to claim 55, and a pharmaceutically acceptable excipient.

60. A method of treating cancer in a subject in need thereof, comprising administering a therapeutically effective amount of the genetically modified T cells according to claim 55.

61. The method according to claim 60, characterized by at least one of:
(i) at least 20% of the administered said T cells are present in the serum at least 4 weeks after administration; and
(ii) the cancer is selected from breast cancer, ovary cancer, gastric cancer, glioblastoma, osteosarcoma, and medulloblastoma.

62. The method according to claim 61, wherein the breast cancer is a Her2 positive cancer.

63. The method according to claim 62, wherein the Her2 positive cancer has a score of 1+, 2+, or 3+ in a Her2 immunohistochemistry test.


SEQUENCE LISTING (46 sequences)

SEQ ID NO: 1 - protein, 45 aa, Artificial Sequence
  Synthesized
VKGKHLCPSPLFPGPSKPFWVLVVVGGVLACYSLLVTVAFIIFWV

SEQ ID NO: 2 - protein, 6 aa, Artificial Sequence
  Synthesized
QDVGPN

SEQ ID NO: 3 - protein, 4 aa, Artificial Sequence
  Synthesized
  SITE (4)..(4): X any amino acid or absent
SASX

SEQ ID NO: 4 - protein, 9 aa, Artificial Sequence
  Synthesized
QQYNTYPFT

SEQ ID NO: 5 - protein, 8 aa, Artificial Sequence
  Synthesized
GFTFNTYA

SEQ ID NO: 6 - protein, 10 aa, Artificial Sequence
  Synthesized
IRSKSNNYAT

SEQ ID NO: 7 - protein, 14 aa, Artificial Sequence
  Synthesized
VTSYYDYDKVLFAY

SEQ ID NO: 8 - protein, 41 aa, Artificial Sequence
  Synthesized
RSKRSRLLHSDYMNMTPRRPGPTRKHYQPYAPPRDFAAYRS

SEQ ID NO: 9 - protein, 47 aa, Artificial Sequence
  Synthesized
RFSVVKRGRKKLLYIFKQPFMRPVQTTQEEDGCSCRFPEEEEGGCEL

SEQ ID NO: 10 - protein, 38 aa, Artificial Sequence
  Synthesized
QVRKAAITSYEKSDGVYTGLSTRNQETYETLKHEKPPQ

SEQ ID NO: 11 - protein, 107 aa, Artificial Sequence
  Synthesized
DIVMTQSPKFMSTSVGDRISVTCKASQDVGPNVAWYQQKPGQSPKPLIYSASYLYNGVPDRFTGSGSGTDFSLTISNVQSDDLAEYFCQQYNTYPFTFGGGTKLEIK

SEQ ID NO: 12 - protein, 120 aa, Artificial Sequence
  Synthesized
EVQLEESGGGLVQPKGSLKLSCAASGFTFNTYAMNWVRQAPGKGLEWIVRIRSKSNNYATYYVDSVKDRFTISRDDSQSMLYLQMNNLKTEDTAMYYCVTSYYDYDKVLFAYWGQGTTVT

SEQ ID NO: 13 - protein, 22 aa, Artificial Sequence
  Synthesized
MDFQVQIFSFLLISASVIMSRG

SEQ ID NO: 14 - protein, 14 aa, Artificial Sequence
  Synthesized
GSTSGSGKSSEGKG

SEQ ID NO: 15 - protein, 434 aa, Artificial Sequence
  Synthesized
MDFQVQIFSFLLISASVIMSRGDIVMTQSPKFMSTSVGDRISVTCKASQDVGPNVAWYQQKPGQSPKPLIYSASYLYNGVPDRFTGSGSGTDFSLTISNVQSDDLAEYFCQQYNTYPFTFGGGTKLEIKGSTSGSGKSSEGKGEVQLEESGGGLVQPKGSLKLSCAASGFTFNTYAMNWVRQAPGKGLEWIVRIRSKSNNYATYYVDSVKDRFTISRDDSQSMLYLQMNNLKTEDTAMYYCVTSYYDYDKVLFAYWGQGTTVTVKGKHLCPSPLFPGPSKPFWVLVVVGGVLACYSLLVTVAFIIFWVRSKRSRLLHSDYMNMTPRRPGPTRKHYQPYAPPRDFAAYRSRFSVVKRGRKKLLYIFKQPFMRPVQTTQEEDGCSCRFPEEEEGGCELQVRKAAITSYEKSDGVYTGLSTRNQETYETLKHEKPPQ

SEQ ID NO: 16 - protein, 24 aa, Artificial Sequence
  Synthesized
TGGAGCCACCCCCAGTTCGAGAAG

SEQ ID NO: 17 - protein, 8 aa, Artificial Sequence
  Synthesized
DYKDDDDK

SEQ ID NO: 18 - protein, 27 aa, Artificial Sequence
  Synthesized
FWVLVVVGGVLACYSLLVTVAFIIFWV

SEQ ID NO: 19 - protein, 48 aa, Artificial Sequence
  Synthesized
AKPTTTPAPRPPTPAPTIASQPLSLRPEACRPAAGGAVHTRGLDFACD

SEQ ID NO: 20 - protein, 59 aa, Artificial Sequence
  Synthesized
FWLPIGCAAFVVVCILGCILICWLTKKKYSSSVHDPNGEYMFMRAVNTAKKSRLTDVTL

SEQ ID NO: 21 - protein, 113 aa, Artificial Sequence
  Synthesized
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 22 - protein, 18 aa, Artificial Sequence
  Synthesized
VKGKHLCPSPLFPGPSKP

SEQ ID NO: 23 - protein, 241 aa, Artificial Sequence
  Synthesized
DIVMTQSPKFMSTSVGDRISVTCKASQDVGPNVAWYQQKPGQSPKPLIYSASYLYNGVPDRFTGSGSGTDFSLTISNVQSDDLAEYFCQQYNTYPFTFGGGTKLEIKGSTSGSGKSSEGKGEVQLEESGGGLVQPKGSLKLSCAASGFTFNTYAMNWVRQAPGKGLEWIVRIRSKSNNYATYYVDSVKDRFTISRDDSQSMLYLQMNNLKTEDTAMYYCVTSYYDYDKVLFAYWGQGTTVT

SEQ ID NO: 24 - protein, 68 aa, Artificial Sequence
  Synthesized
FWVLVVVGGVLACYSLLVTVAFIIFWVRSKRSRLLHSDYMNMTPRRPGPTRKHYQPYAPPRDFAAYRS

SEQ ID NO: 25 - protein, 21 aa, Artificial Sequence
  Synthesized
FWLPIGCAAFVVVCILGCILI

SEQ ID NO: 26 - protein, 21 aa, Artificial Sequence
  Synthesized
MALPVTALLLPLALLLHAARP

SEQ ID NO: 27 - protein, 509 aa, Artificial Sequence
  Synthesized
MDFQVQIFSFLLISASVIMSRGDIVMTQSPKFMSTSVGDRISVTCKASQDVGPNVAWYQQKPGQSPKPLIYSASYLYNGVPDRFTGSGSGTDFSLTISNVQSDDLAEYFCQQYNTYPFTFGGGTKLEIKGSTSGSGKSSEGKGEVQLEESGGGLVQPKGSLKLSCAASGFTFNTYAMNWVRQAPGKGLEWIVRIRSKSNNYATYYVDSVKDRFTISRDDSQSMLYLQMNNLKTEDTAMYYCVTSYYDYDKVLFAYWGQGTTVTVKGKHLCPSPLFPGPSKPFWVLVVVGGVLACYSLLVTVAFIIFWVRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 28 - protein, 538 aa, Artificial Sequence
  Synthesized
MALPVTALLLPLALLLHAARPDIVMTQSPKFMSTSVGDRISVTCKASQDVGPNVAWYQQKPGQSPKPLIYSASYLYNGVPDRFTGSGSGTDFSLTISNVQSDDLAEYFCQQYNTYPFTFGGGTKLEIKGSTSGSGKSSEGKGEVQLEESGGGLVQPKGSLKLSCAASGFTFNTYAMNWVRQAPGKGLEWIVRIRSKSNNYATYYVDSVKDRFTISRDDSQSMLYLQMNNLKTEDTAMYYCVTSYYDYDKVLFAYWGQGTTVTAKPTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 29 - protein, 529 aa, Artificial Sequence
  Synthesized
MALPVTALLLPLALLLHAARPDIVMTQSPKFMSTSVGDRISVTCKASQDVGPNVAWYQQKPGQSPKPLIYSASYLYNGVPDRFTGSGSGTDFSLTISNVQSDDLAEYFCQQYNTYPFTFGGGTKLEIKGSTSGSGKSSEGKGEVQLEESGGGLVQPKGSLKLSCAASGFTFNTYAMNWVRQAPGKGLEWIVRIRSKSNNYATYYVDSVKDRFTISRDDSQSMLYLQMNNLKTEDTAMYYCVTSYYDYDKVLFAYWGQGTTVTAKPTTTPAPRPPTPAPTIASQPLSLRPEACRPAAGGAVHTRGLDFACDFWLPIGCAAFVVVCILGCILICWLTKKKYSSSVHDPNGEYMFMRAVNTAKKSRLTDVTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 30 - DNA, 321 bp, Artificial Sequence
  Synthesized
gatattgtgatgacccagtctccaaaattcatgtccacatcagtaggagacaggatcagcgtcacctgcaaggccagtcaagatgtgggtcctaatgtagcctggtatcaacagaaaccagggcaatctcctaaaccactgatttactcggcatcctacctatataatggagtccctgatcgcttcacaggcagtggatctgggacagatttctctctcaccatcagcaatgtgcagtctgatgacttggcagagtatttctgtcagcaatataacacctatccgttcacgttcggagggggcaccaagctggaaatcaaa

SEQ ID NO: 31 - DNA, 360 bp, Artificial Sequence
  Synthesized
gaggtgcagctggaggagtctggtggaggattggtgcagcctaaagggtcattgaaactctcatgtgcagcctctggattcaccttcaatacctacgccatgaactgggtccgccaggctccaggaaagggtttggaatggattgttcgcataagaagtaaaagtaataattatgcaacatattatgtcgattcagtgaaagacaggttcaccatctccagagatgattcacaaagcatgctctatctgcaaatgaacaacttgaaaactgaggacacagccatgtattactgtgtgacttcttactatgattacgacaaggtcctgtttgcttactggggccaagggaccacggtcacc

SEQ ID NO: 32 - DNA, 42 bp, Artificial Sequence
  Synthesized
gggtcgacttccggtagcggcaaatcctctgaaggcaaaggt

SEQ ID NO: 33 - DNA, 723 bp, Artificial Sequence
  Synthesized
gatattgtgatgacccagtctccaaaattcatgtccacatcagtaggagacaggatcagcgtcacctgcaaggccagtcaagatgtgggtcctaatgtagcctggtatcaacagaaaccagggcaatctcctaaaccactgatttactcggcatcctacctatataatggagtccctgatcgcttcacaggcagtggatctgggacagatttctctctcaccatcagcaatgtgcagtctgatgacttggcagagtatttctgtcagcaatataacacctatccgttcacgttcggagggggcaccaagctggaaatcaaagggtcgacttccggtagcggcaaatcctctgaaggcaaaggtgaggtgcagctggaggagtctggtggaggattggtgcagcctaaagggtcattgaaactctcatgtgcagcctctggattcaccttcaatacctacgccatgaactgggtccgccaggctccaggaaagggtttggaatggattgttcgcataagaagtaaaagtaataattatgcaacatattatgtcgattcagtgaaagacaggttcaccatctccagagatgattcacaaagcatgctctatctgcaaatgaacaacttgaaaactgaggacacagccatgtattactgtgtgacttcttactatgattacgacaaggtcctgtttgcttactggggccaagggaccacggtcacc

SEQ ID NO: 34 - DNA, 141 bp, Artificial Sequence
  Synthesized
cgtttctctgttgttaaacggggcagaaagaagctcctgtatatattcaaacaaccatttatgagaccagtacaaactactcaagaggaagatggctgtagctgccgatttccagaagaagaagaaggaggatgtgaactg

SEQ ID NO: 35 - DNA, 204 bp, Artificial Sequence
  Synthesized
ttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatagatct

SEQ ID NO: 36 - DNA, 339 bp, Artificial Sequence
  Synthesized
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 37 - DNA, 114 bp, Artificial Sequence
  Synthesized
caagtgcgaaaggcagctataaccagctatgagaaatcagatggtgtttacacgggcctgagcaccaggaaccaggagacttacgagactctgaagcatgagaaaccaccacag

SEQ ID NO: 38 - DNA, 66 bp, Artificial Sequence
  Synthesized
atggattttcaggtgcagattttcagcttcctgctaatcagtgcctcagtcataatgtctagagga

SEQ ID NO: 39 - DNA, 63 bp, Artificial Sequence
  Synthesized
atggccttaccagtgaccgccttgctcctgccgctggccttgctgctccacgccgccaggccg

SEQ ID NO: 40 - DNA, 54 bp, Artificial Sequence
  Synthesized
gtgaaagggaaacacctttgtccaagtcccctatttcccggaccttctaagccc

SEQ ID NO: 41 - DNA, 144 bp, Artificial Sequence
  Synthesized
gcaaagccaacaaccactccagccccccggcctccaacgcctgcgccaaccattgcatcacaaccgctcagcctgagacccgaagcgtgcagacccgccgcagggggagcggtccatacacgggggctggactttgcttgtgac

SEQ ID NO: 42 - DNA, 177 bp, Artificial Sequence
  Synthesized
ttttggcttcccattggttgcgcggcgtttgtcgtagtctgcatcctgggatgcattctcatttgttggttgacgaagaagaaatatagttcctcagtgcatgatccaaacggtgaatacatgtttatgagggccgtaaacacagctaagaagtctcgcttgactgacgttacactg

SEQ ID NO: 43 - DNA, 1305 bp, Artificial Sequence
  Synthesized
atggattttcaggtgcagattttcagcttcctgctaatcagtgcctcagtcataatgtctagaggagatattgtgatgacccagtctccaaaattcatgtccacatcagtaggagacaggatcagcgtcacctgcaaggccagtcaagatgtgggtcctaatgtagcctggtatcaacagaaaccagggcaatctcctaaaccactgatttactcggcatcctacctatataatggagtccctgatcgcttcacaggcagtggatctgggacagatttctctctcaccatcagcaatgtgcagtctgatgacttggcagagtatttctgtcagcaatataacacctatccgttcacgttcggagggggcaccaagctggaaatcaaagggtcgacttccggtagcggcaaatcctctgaaggcaaaggtgaggtgcagctggaggagtctggtggaggattggtgcagcctaaagggtcattgaaactctcatgtgcagcctctggattcaccttcaatacctacgccatgaactgggtccgccaggctccaggaaagggtttggaatggattgttcgcataagaagtaaaagtaataattatgcaacatattatgtcgattcagtgaaagacaggttcaccatctccagagatgattcacaaagcatgctctatctgcaaatgaacaacttgaaaactgaggacacagccatgtattactgtgtgacttcttactatgattacgacaaggtcctgtttgcttactggggccaagggaccacggtcaccgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatagatctcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgcaagtgcgaaaggcagctataaccagctatgagaaatcagatggtgtttacacgggcctgagcaccaggaaccaggagacttacgagactctgaagcatgagaaaccaccacagtag

SEQ ID NO: 44 - DNA, 1617 bp, Artificial Sequence
  Synthesized
atggccttaccagtgaccgccttgctcctgccgctggccttgctgctccacgccgccaggccggatattgtgatgacccagtctccaaaattcatgtccacatcagtaggagacaggatcagcgtcacctgcaaggccagtcaagatgtgggtcctaatgtagcctggtatcaacagaaaccagggcaatctcctaaaccactgatttactcggcatcctacctatataatggagtccctgatcgcttcacaggcagtggatctgggacagatttctctctcaccatcagcaatgtgcagtctgatgacttggcagagtatttctgtcagcaatataacacctatccgttcacgttcggagggggcaccaagctggaaatcaaagggtcgacttccggtagcggcaaatcctctgaaggcaaaggtgaggtgcagctggaggagtctggtggaggattggtgcagcctaaagggtcattgaaactctcatgtgcagcctctggattcaccttcaatacctacgccatgaactgggtccgccaggctccaggaaagggtttggaatggattgttcgcataagaagtaaaagtaataattatgcaacatattatgtcgattcagtgaaagacaggttcaccatctccagagatgattcacaaagcatgctctatctgcaaatgaacaacttgaaaactgaggacacagccatgtattactgtgtgacttcttactatgattacgacaaggtcctgtttgcttactggggccaagggaccacggtcaccgcaaagccaacaaccactccagccccccggcctccaacgcctgcgccaaccattgcatcacaaccgctcagcctgagacccgaagcgtgcagacccgccgcagggggagcggtccatacacgggggctggactttgcttgtgacttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatagatctcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag

SEQ ID NO: 45 - DNA, 1617 bp, Artificial Sequence; identical to SEQ ID NO: 44
  Synthesized

SEQ ID NO: 46 - DNA, 1590 bp, Artificial Sequence
  Synthesized
atggccttaccagtgaccgccttgctcctgccgctggccttgctgctccacgccgccaggccggatattgtgatgacccagtctccaaaattcatgtccacatcagtaggagacaggatcagcgtcacctgcaaggccagtcaagatgtgggtcctaatgtagcctggtatcaacagaaaccagggcaatctcctaaaccactgatttactcggcatcctacctatataatggagtccctgatcgcttcacaggcagtggatctgggacagatttctctctcaccatcagcaatgtgcagtctgatgacttggcagagtatttctgtcagcaatataacacctatccgttcacgttcggagggggcaccaagctggaaatcaaagggtcgacttccggtagcggcaaatcctctgaaggcaaaggtgaggtgcagctggaggagtctggtggaggattggtgcagcctaaagggtcattgaaactctcatgtgcagcctctggattcaccttcaatacctacgccatgaactgggtccgccaggctccaggaaagggtttggaatggattgttcgcataagaagtaaaagtaataattatgcaacatattatgtcgattcagtgaaagacaggttcaccatctccagagatgattcacaaagcatgctctatctgcaaatgaacaacttgaaaactgaggacacagccatgtattactgtgtgacttcttactatgattacgacaaggtcctgtttgcttactggggccaagggaccacggtcaccgcaaagccaacaaccactccagccccccggcctccaacgcctgcgccaaccattgcatcacaaccgctcagcctgagacccgaagcgtgcagacccgccgcagggggagcggtccatacacgggggctggactttgcttgtgacttttggcttcccattggttgcgcggcgtttgtcgtagtctgcatcctgggatgcattctcatttgttggttgacgaagaagaaatatagttcctcagtgcatgatccaaacggtgaatacatgtttatgagggccgtaaacacagctaagaagtctcgcttgactgacgttacactgcgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctag